(19) United States
(12) Patent Application Publication
(10) Pub. No.: US 2024/0358339 A1
(43) Pub. Date: Oct. 31, 2024

(54) METHODS AND SYSTEMS FOR IMAGING

(71) Applicant: SHANGHAI UNITED IMAGING HEALTHCARE CO., LTD., Shanghai (CN)

(72) Inventor: Yangyi LIU, Shanghai (CN)

(73) Assignee: SHANGHAI UNITED IMAGING HEALTHCARE CO., LTD., Shanghai (CN)

(21) Appl. No.: 18/765,321

(22) Filed: Jul. 7, 2024

Related U.S. Application Data
(63) Continuation of application No. PCT/CN2023/102968, filed on Jun. 27, 2023.

(30) Foreign Application Priority Data
Jun. 27, 2022 (CN) 202210737557.8

(51) Int. Cl. A61B 6/00; A61B 6/03; A61B 6/42

(52) U.S. Cl. CPC A61B 6/5282; A61B 6/032; A61B 6/5229; A61B 6/4241

(57) ABSTRACT

The present disclosure is related to methods and systems for imaging. The method may include obtaining current information of a target subject. The current information may include information relating to current radiation attenuation of the target object. The method may also include obtaining reference information of the target subject. The reference information may include information relating to historical radiation attenuation of the target object. The method may also include determining at least one current parameter of the target subject based on the current information and the reference information. The method may also include obtaining a target image of the target subject based on the at least one current parameter.

### CROSS-REFERENCE TO RELATED APPLICATIONS

This application is a Continuation of International Application No. PCT/CN2023/102968 filed on Jun. 27, 2023, which claims priority to Chinese Patent Application No. 202210737557.8, filed on Jun. 27, 2022, the entire contents of each of which are hereby incorporated by reference.

### TECHNICAL FIELD

The present disclosure generally relates to the field of medical imaging, and more particularly, relates to methods and systems for imaging.

### BACKGROUND

Photon-Counting Computed Tomography (PCCT) has been widely used in medical imaging due to its advantages of enabling material composition analysis, reducing patient radiation dose, improving an accuracy of quantitative CT analysis, achieving ultra-high spatial resolution, etc. In PCCT imaging, an operator can obtain quantitative results of material(s) of a target subject from tomographic images and make reliable qualitative determinations based on the quantitative results. However, an accuracy of the quantitative results of material decomposition depends on a scanning system, scanning parameters, and a subsequent processing of data.

Thus, it is desirable to provide methods and systems for imaging using a PCCT device, so as to determine current scanning parameter(s) and/or current processing parameter(s) of the target subject and improve an accuracy of a quantitative result of material decomposition.

### SUMMARY

An aspect of the present disclosure provides a method implemented on at least one machine each of which has at least one processor and at least one storage device for imaging. The method may include obtaining current information of a target subject. The current information including information relating to current radiation attenuation of the target object. The method may also include obtaining reference information of the target subject. The reference information including information relating to historical radiation attenuation of the target object. The method may further include determining at least one current parameter of the target subject based on the current information and the reference information and obtaining a target image of the target subject based on the at least one current parameter.

In some embodiments, the current information may include information relating to at least one of a current body shape of the target subject, a current body fat rate of the target subject, or a current 3D model of the target object; and the reference information may include information relating to at least one of a reference body shape of the target subject, a reference body fat rate of the target subject, or a reference 3D model of the target object.

In some embodiments, the method may further include obtaining a comparison result by comparing the target image of the target subject with a historical image corresponding to the reference information.

In some embodiments, the determining at least one current parameter of the target subject based on the current information and the reference information may include obtaining at least one historical parameter corresponding to the reference information and determining the at least one current parameter based on the current information, the reference information, and the at least one historical parameter.

In some embodiments, the determining the at least one current parameter based on the current information, the reference information, and the at least one historical parameter may include obtaining information variation between the current information and the reference information and determining the at least one current parameter based on the information variation and the at least one historical parameter. The information variation may be related to radiation attenuation variation of the target subject.

In some embodiments, the determining the at least one current parameter based on the information variation and the at least one historical parameter may include obtaining at least one initial parameter based on the at least one historical parameter; updating the at least one initial parameter iteratively based on the information variation until a predicted image generated based on the at least one updated parameter and the historical image satisfy a preset condition and determining the at least one updated parameter as the least one current parameter.

In some embodiments, the preset condition may include that the predicted image has a same or a substantially similar confidence degree with the historical image or a background region of the predicted image has at least one substantially same feature value as a background region of the historical image.

In some embodiments, the at least one historical parameter may include at least one of a historical scanning parameter or a historical processing parameter, and the at least one current parameter may include at least one of a current scanning parameter or a current processing parameter.

In some embodiments, the determining the at least one current parameter based on the current information, the reference information, and the at least one historical parameter may include determining the current scanning parameter based on the current information, the reference information, and the historical scanning parameter.

In some embodiments, the obtaining a target image of the target subject based on the at least one current parameter may include obtaining scan data by scanning the target subject based on the current scanning parameter and obtaining the target image of the target subject by processing the scan data based on the historical processing parameter.

In some embodiments, the determining the at least one current parameter based on the current information, the reference information, and the at least one historical parameter may include determining the current processing parameter based on the current information, the reference information, and the historical processing parameter.

In some embodiments, the obtaining a target image of the target subject based on the at least one current parameter may include obtaining scan data by scanning the target subject based on the historical scanning parameter and obtaining the target image of the target subject by processing the scan data based on the current processing parameter.

In some embodiments, the obtaining a target image of the target subject based on the at least one current parameter may include obtaining scan data by scanning the target subject based on the current scanning parameter or a preset scanning parameter, and obtaining the target image of the target subject by processing the scan data based on the current processing parameter or a preset processing parameter.

Another aspect of the present disclosure provides a method implemented on at least one machine each of which has at least one processor and at least one storage device for image processing. The method may comprise building a personal database of a target subject. The personal database may be configured to store historical scanning data of the target subject. The historical scanning data may include at least one historical parameter of the target subject, at least one historical image of the target subject, and reference information of the target subject. The reference information may include information relating to historical radiation attenuation of the target object. The method may also include obtaining current scanning data of the target subject. The method may further include generating a quantitative comparison result by comparing the current scanning data and the historical scanning data.

In some embodiments, the current scanning data includes a target image, and the obtaining current scanning data of the target subject may include obtaining current information of the target subject, determining at least one current parameter of the target subject based on the current information, the reference information, and the at least one historical parameter, and obtaining the target image of the target subject based on the at least one current parameter. The current information may include information relating to current radiation attenuation of the target object.

In some embodiments, the determining at least one current parameter of the target subject based on the current information, the reference information, and the at least one historical parameter may include obtaining information variation between the current information and the reference information and determining the at least one current parameter based on the information variation and the at least one historical parameter. The information variation being related to radiation attenuation variation of the target subject.

Another aspect of the present disclosure provides a method implemented on at least one machine each of which has at least one processor and at least one storage device for imaging using a photon counting computed tomography device. The method may include obtaining current information of a target subject and obtaining reference information and at least one historical parameter of the target subject. The current information may include information relating to current radiation attenuation of the target object. The reference information may include information relating to historical radiation attenuation of the target object. The at least one historical parameter may include at least one of a historical scanning parameter or a historical processing parameter. The method may also include determining a current scanning parameter and/or a current processing parameter of the target subject based on the current information, the reference information, and the at least one historical parameter.

Another aspect of the present disclosure provides a system for imaging using a photon counting computed tomography device. The system may include at least one storage device storing a set of instructions and at least one processor in communication with the storage device. When executing the set of instructions, the at least one processor may be configured to cause the system to perform operations. The operations may include obtaining current information of a target subject, obtaining reference information of the target subject, determining at least one current parameter of the target subject based on the current information and the reference information, and obtaining a target image of the target subject based on the at least one current paramete. The current information may include information relating to current radiation attenuation of the target object. The reference information may include information relating to historical radiation attenuation of the target object.

Another aspect of the present disclosure provides a system for imaging using a photon counting computed tomography device. The system includes a first obtaining module configured to obtain current information of a target subject and reference information of the target subject, a first determining module configured to determine at least one current parameter of the target subject based on the current information and the reference information, and a first scanning module configured to obtain a target image of the target subject based on the at least one current parameter. The current information may include information relating to current radiation attenuation of the target object. The reference information may include information relating to historical radiation attenuation of the target object.

Another aspect of the present disclosure provides a non-transitory computer readable medium storing instructions. The instructions, when executed by at least one processor, may cause the at least one processor to implement a method. The method may include obtaining current information of a target subject, obtaining reference information of the target subject, determining at least one current parameter of the target subject based on the current information and the reference information, and obtaining a target image of the target subject based on the at least one current parameter. The current information may include information relating to current radiation attenuation of the target object. The reference information may include information relating to historical radiation attenuation of the target object.

Another aspect of the present disclosure provides a system for imaging using a photon counting computed tomography device. The system may include at least one storage device storing a set of instructions and at least one processor in communication with the storage device. When executing the set of instructions, the at least one processor may be configured to cause the system to perform operations. The operations may include obtaining current information of a target subject and obtaining reference information and at least one historical parameter of the target subject. The at least one historical parameter may include at least one of a historical scanning parameter or a historical processing parameter. The operations may also include determining a current scanning parameter and/or a current processing parameter of the target subject based on the current information, the reference information, and the at least one historical parameter. The current information may include information relating to current radiation attenuation of the target object. The reference information may include information relating to historical radiation attenuation of the target object.

Another aspect of the present disclosure provides a system for imaging using a photon counting computed tomography device. The system may include an obtaining module configured to obtain current information of a target subject and obtain reference information and at least one historical parameter of the target subject. The at least one historical parameter may include at least one of a historical scanning parameter or a historical processing parameter. The system may also include a determining module configured to determine a current scanning parameter and/or a current processing parameter of the target subject based on the current information, the reference information, and the at least one historical parameter. The system may also include a scanning module configured to scan the target subject based on the current scanning parameter or a preset scanning parameter and obtain an output of a detector of the photon counting computed tomography device. The system may further include a data processing module configured to obtain a target image of the target subject by processing the output of the detector based on the current processing parameter. The current information may include information relating to current radiation attenuation of the target object. The reference information may include information relating to historical radiation attenuation of the target object.

Another yet aspect of the present disclosure provides a non-transitory computer readable medium storing instructions. The instructions, when executed by at least one processor, may cause the at least one processor to implement a method. The method may include obtaining current information of a target subject and obtaining reference information and at least one historical parameter of the target subject. The at least one historical parameter may include at least one of a historical scanning parameter or a historical processing parameter. The method may also include determining a current scanning parameter and/or a current processing parameter of the target subject based on the current information, the reference information, and the at least one historical parameter. The current information may include information relating to current radiation attenuation of the target object. The reference information may include information relating to historical radiation attenuation of the target object.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities, and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. The drawings are not to scale. These embodiments are non-limiting exemplary embodiments, in which like reference numerals represent similar structures throughout the several views of the drawings, and wherein:

FIG. 1 is a schematic diagram illustrating an exemplary application scenario of an imaging system according to some embodiments of the present disclosure;

FIG. 2 is a schematic diagram illustrating exemplary hardware and/or software components of an exemplary computing device according to some embodiments of the present disclosure;

FIG. 3 is a block diagram illustrating an exemplary imaging system according to some embodiments of the present disclosure;

FIG. 4 is a block diagram illustrating an exemplary imaging system according to some embodiments of the present disclosure;

FIG. 5 is a flowchart illustrating an exemplary imaging process according to some embodiments of the present disclosure;

FIG. 6 is a flowchart illustrating an exemplary process of determining at least one current parameter according to some embodiments of the present disclosure;

FIG. 7 is a flowchart illustrating an exemplary process of determining a current scanning parameter according to some embodiments of the present disclosure;

FIG. 8 is a flowchart illustrating an exemplary process of determining a current processing parameter according to some embodiments of the present disclosure;

FIG. 9 is a flowchart illustrating an exemplary imaging process according to some embodiments of the present disclosure;

FIG. 10 is a flowchart illustrating an exemplary imaging process according to some embodiments of the present disclosure;

FIG. 11 is a flowchart illustrating an exemplary imaging process according to some embodiments of the present disclosure; and

FIG. 12 is a flowchart illustrating an exemplary imaging process according to some embodiments of the present disclosure.

### DETAILED DESCRIPTION

In order to more clearly illustrate the technical solutions of the embodiments of the present disclosure, the following briefly introduces the drawings that need to be used in the description of the embodiments. Obviously, the accompanying drawings in the following description are only some examples or embodiments of the present disclosure, for those skilled in the art, the present disclosure may also be applied to other similar scenarios according to these drawings without any creative effort. Unless obviously obtained from the context or the context illustrates otherwise, the same numeral in the drawings refers to the same structure or operation.

It will be understood that the term “system,” “engine,” “unit,” “module,” and/or “block” used herein are one method to distinguish different components, elements, parts, sections or assembly of different levels in ascending order. However, the terms may be displaced by another expression if they achieve the same purpose.

The terminology used herein is for the purpose of describing particular example embodiments only and is not intended to be limiting. As used herein, the singular forms “a,” “an,” and “the” may be intended to include the plural forms as well, unless the context clearly indicates otherwise. It will be further understood that the terms “comprise,” “comprises,” and/or “comprising,” “include,” “includes,” and/or “including” when used in this disclosure, specify the presence of stated features, integers, steps, operations, elements, and/or components, but do not preclude the presence or addition of one or more other features, integers, steps, operations, elements, components, and/or groups thereof.

The flowcharts used in the present disclosure illustrate operations that systems implement according to some embodiments in the present disclosure. Related descriptions are intended to facilitate a better understanding of methods and/or systems for medical imaging. It is to be expressly understood, the operations of the flowchart may be implemented not in order. Conversely, the operations may be implemented in an inverted order, or simultaneously. Moreover, one or more other operations may be added to the flowcharts. One or more operations may be removed from the flowcharts.

CT scanning using a photon counting computed tomography device may provide a function of material decomposition. An operator may obtain quantitative results of material decomposition of a target subject with certain accuracy based on a tomography image obtained using PCCT. The operator may obtain a reliable qualitative determination in a specific clinical scenario (e.g., a nature of a tumor, a rehabilitation progress of a patient, etc.) based on the quantitative results, thereby reducing the requirements for clinical experience of a physician. However, an accuracy degree of the quantitative results may depend on a scanning system, a scanning parameter, a subsequent processing progress of data simultaneously.

Merely by way of example, attenuation information of a target subject may be changed due to a change of cancer cells, a body shape of the target subject, etc., before and after performing a radiotherapy on a patient. Therefore, different scanning parameters may be used before and after the radiotherapy to analyze cancer cell information of the patient based on the medical image before and after the radiotherapy, thereby keeping X-ray(s) emitted on a cancer portion to be consistent during the two times of scans to obtain an accurate quantitative analysis result to facilitate the analysis of a diffusion level and/or a size of the cancer cells.

The embodiments of the present disclosure provide a method and system for imaging using a photon counting computed tomography device. Reference information, a historical scanning parameter, and a historical processing parameter may be obtained from a personal database of each target subject, and may be used (with current information of the target subject) to determine current scanning parameter(s) and/or current processing parameter(s) of the target subject. A target image of the target subject may be obtained based on the current scanning parameter(s) and/or the current processing parameter(s), and a comparison result may be obtained by comparing the target image of the target subject with a historical image corresponding to the reference information, so that scanning results of each scan in series scans of the target subject may be quantitatively comparable, which is convenient for tracking a change of a condition of the target subject, thereby improving diagnosis efficiency and an accuracy of a diagnosis result.

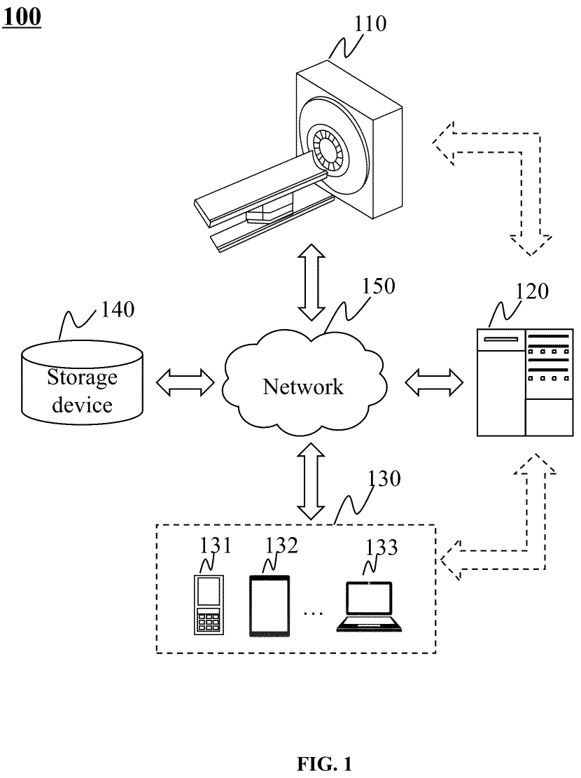

FIG. 1 is a schematic diagram illustrating an exemplary application scenario of an imaging system according to some embodiments of the present disclosure.

As illustrated in FIG. 1, an imaging system 100 may include an imaging device 110, a processing device 120, one or more terminal devices 130, a storage device 140, and a network 150. The components of the imaging system 100 may be connected in one or more of various ways. Merely by way of example, as illustrated in FIG. 1, the imaging device 110 may be connected to the processing device 120 through the network 150. As another example, the imaging device 110 may be connected to the processing device 120 directly, which is as indicated by the bi-directional arrow in dotted lines linking the imaging device 110 and the processing device 120. As a further example, the storage device 140 may be connected to the processing device 120 directly (not shown in FIG. 1) or through the network 150. As another further example, the one or more terminal devices 130 may be connected to the processing device 120 directly (as indicated by the bi-directional arrow in dotted lines linking the terminal device(s) 130 and the processing device 120) or through the network 150.

The imaging device 110 may scan a target subject located within its detection region and generate scanning data (e.g., an output of a detector, an image) relating to the target subject. In some embodiments, the target subject may include a biological subject and/or a non-biological subject. For example, the target subject may include a specific portion of a body, such as head, chest, stomach, or the like, or any combination thereof. As another example, the target subject may be or include animate or inanimate, organic and/or inorganic substances of man-made composition.

In some embodiments, the imaging device 110 may include a non-invasive biological imaging device for disease diagnosis or research purposes. For example, the imaging device 110 may include a single-modality scanner and/or a multi-modality scanner. The single-modality scanner may include, for example, an ultrasonic scanner, an X-ray scanner, a Computed Tomography (CT) scanner, a Magnetic Resonance Imaging (MRI) scanner, an ultrasonic examination instrument, a Positron Emission Computed Tomography (PET) scanner, an optical coherence tomography (OCT) scanner, an ultrasound (US) scanner, an intravascular ultrasound (IVUS) scanner, a near infrared spectroscopy (NIRS) scanner, a far infrared (FIR) scanner, etc.

The multi-modality scanner may include, for example, an X-ray imaging-MRI (X-ray-MRI) scanner, a PET-X-ray imaging (PET-X ray) scanner, a Single-Photon Emission Computed Tomography-X-ray imaging (SPECT-MRI) scanner, a PET-CT scanner, a Digital subtraction angiography-MRI (DSA-MRI) scanner, etc. The scanners mentioned above are merely for illustration purposes, which is not limited to the scope of the present disclosure. As illustrated in the present disclosure, the term “imaging modality” or “modality” broadly refers to an imaging method or technique for collecting, generating, processing and/or analyzing imaging information of a target subject.

In some embodiments, the imaging device 110 may include modules and/or components used to perform imaging and/or related analysis. For example, the imaging device 110 may include a ray generating device, an accessary device, and an imaging device. The ray generating device may refer to a device used to generate and control rays (e.g., X-rays). The accessary device may refer to a facility used to satisfy requirements of clinical diagnosis and treatment. For example, the accessary device may include a mechanical device such as an examining table, a clinical table, a table with draft tube, a photography table, etc., various supports, a suspension device, a braking device, a grid, a keeping device, a shutter, etc. In some embodiments, the ray generating device may be in various forms. For example, a digital imaging device may include a detector, a computer system and image processing software, etc. Another imaging device may include a phosphor screen, a cassette, an image intensifier, a video TV, etc.

In some embodiments, data (e.g., a historical image, a target image of a target subject, etc.) acquired by the imaging device 110 may be transmitted to the processing device 120 for further analysis (e.g., a comparison result is obtained by comparing a historical image of a target subject with a target image). Alternatively, data acquired by the imaging device 110 may be transmitted to a terminal device (e.g., the terminal device 130) for display and/or a storage device (e.g., the storage device 140) for storage.

The processing device 120 may process data and/or information obtained from the imaging device 110, the terminal device 130, the storage device 140, and/or other storage devices. For example, the processing device 120 may obtain current information, reference information, a historical scanning parameter, and a historical processing parameter from the terminal device 130 or the storage device 140, and determine a current scanning parameter and/or a current processing parameter of the target subject based on the current information, the reference information, the historical scanning parameter, and the historical processing parameter. As another example, the processing device 120 may obtain the target image of the target subject from the imaging device 110, compare the target image with a historical image of the target subject, and output a comparison result.

In some embodiments, the processing device 120 may be a single server or a server group. The server group may be centralized or distributed. In some embodiments, the processing device 120 may be local to or remote from the image processing system 100. In some embodiments, the processing device 120 may be implemented on a cloud platform. Merely by way of example, the cloud platform may include a private cloud, a public cloud, a hybrid cloud, a community cloud, a distributed cloud, an inter-cloud, a multi-cloud, or the like, or any combination thereof.

In some embodiments, the processing device 120 may be implemented on a computing device. In some embodiments, the processing device 120 may be implemented on a terminal (e.g., the terminal device 130). In some embodiments, the processing device 120 may be implemented on an imaging device (e.g., the imaging device 110). For example, the processing device 120 may be integrated in the terminal device 130 and/or the imaging device 110.

The terminal device 130 may be connected to the imaging device 110 and/or the processing device 120 to input or output information and/or data. For example, a user may interact with the imaging device 110 via the terminal device 130 to control one or more components (e.g., input patient information, select a parameter determination mode (e.g., automatic determination, manual input, or semi-automatic determination), adjust at least one of current parameter (e.g., adjust a current scanning parameter and/or a current processing parameter), etc.) of the imaging device 110. As another example, the imaging device 110 may transmit the medical image (e.g., a target image) and/or the quantitative analysis result (e.g., a comparison result between a historical image of a target subject and a target image) to the terminal device 130 to display the medical image and/or the quantitative analysis result to the user.

In some embodiments, the terminal device 130 may include a mobile device 131, a tablet computer 132, a laptop computer 133, or the like, or any combination thereof. In some embodiments, the mobile device 131 may include a smart home device, a wearable device, a smart mobile device, a virtual reality device, an augmented reality device, or the like, or any combination thereof.

In some embodiments, one or more terminal devices 130 may remotely operate the imaging device 110. In some embodiments, the terminal device 130 may operate the imaging device 110 via a wireless connection. In some embodiments, one or more terminal devices 130 may be part of the processing device 120. In some embodiments, the terminal device 130 may be omitted.

The storage device 140 may store data and/or instructions. In some embodiments, the storage device 140 may store data obtained from the terminal device 130 and/or the processing device 120. For example, the storage device 140 may store a target count of energy bins, scan protocols, etc. In some embodiments, the storage device 140 may store data and/or instructions that the processing device 120 may execute or use to perform exemplary methods described in the present disclosure.

In some embodiments, the storage device 140 may include a mass storage device, a removable storage device, a volatile read-and-write memory, a read-only memory (ROM), or the like, or any combination thereof. Exemplary mass storage devices may include a magnetic disk, an optical disk, a solid-state drive, etc. Exemplary removable storage devices may include a flash drive, a floppy disk, an optical disk, a memory card, a zip disk, a magnetic tape, etc. Exemplary volatile read-and-write memory may include a random-access memory (RAM). In some embodiments, the storage device 140 may be part of the processing device 120.

The network 150 may include any suitable network that can facilitate the exchange of information and/or data for the imaging system 100. In some embodiments, one or more components of the imaging system 100 (e.g., the imaging device 110, one or more terminal devices 130, the processing device 120, or the storage device 140) may communicate information and/or data with one or more other components of the imaging system 100 via the network 150.

In some embodiments, the network 140 may be any type of wired or wireless network, or a combination thereof. The network 150 may be and/or include a public network (e.g., the Internet), a private network (e.g., a local area network (LAN), a wide area network (WAN)), etc.), a wired network (e.g., an Ethernet network), a wireless network (e.g., an 802.11 network, a Wi-Fi network), a cellular network (e.g., a Long Term Evolution (LTE) network), a frame relay network, a virtual private network (“VPN”), a satellite network, a telephone network, routers, hubs, switches, server computers, and/or any combination thereof. In some embodiments, the network 120 may include one or more network access points.

It should be noted that the above description of the imaging system 100 is intended to be illustrative, and not to limit the scope of the present disclosure. Many alternatives, modifications, and variations will be apparent to those skilled in the art. However, these alternatives, modifications, and variations may not depart from the scope of the present disclosure. For example, the imaging device 110, the processing device 120, and the terminal device 130 may share the storage device 140, or have their own storage device.

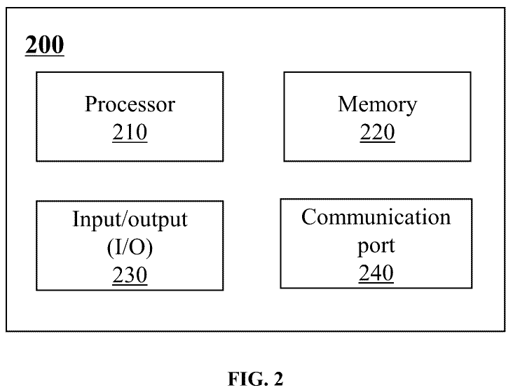

FIG. 2 is a schematic diagram illustrating exemplary hardware and/or software components of an exemplary computing device according to some embodiments of the present disclosure.

As shown in FIG. 2, in some embodiments, the computing device 200 may include a processor 210, a memory 220, an input/output (I/O) 230, and a communication port 240.

The processor 210 may execute computer instructions (e.g., program code) and perform functions of the processing device 120 according to the method(s) described herein. The computer instructions may include, for example, routines, programs, objects, components, data structures, procedures, modules, and functions, which perform particular functions described herein. For example, the processor 210 may process data of the imaging device 110, the terminal device 130, the storage device 140, and/or any other component in the imaging system 100. In some embodiments, the processor 210 may include at least one hardware processor, such as a microcontroller, a microprocessor, a reduced instruction set computer (RISC), an application specific integrated circuit (ASIC), an application specific instruction set processor (ASIP), a central processing unit (CPU), a graphics processing unit (GPU), a physical processing unit (PPU), microcontroller unit, a digital signal processor (DSP), a field programmable gate array (FPGA), a high-order RISC Machine (ARM), a programmable logic device (PLD), any circuit or processor or similar capable of performing at least one function, or any combination thereof.

Merely for illustration, only one processor is described in the computing device 200. However, it should be noted that the computing device 200 in the present disclosure may also include multiple processors, thus operations and/or method steps that are performed by one processor as described in the present disclosure may also be jointly or separately performed by the multiple processors. For example, if in the present disclosure the processor of the computing device 200 executes both operations A and B, it should be understood that operation A and operation B may also be performed by two or more different processors jointly or separately in the computing device 200 (e.g., a first processor executes operation A and a second processor executes operation B, or the first and second processors jointly execute operations A and B).

The memory 220 may store data/information obtained from the imaging device 110, the terminal device 130, the storage device 140, and/or any other component in the imaging system 100. In some embodiments, the memory 220 may include a mass storage, a removable storage, a volatile read-write memory, a read-only memory (ROM), or any combination thereof. In some embodiments, the memory 220 may store at least one program and/or instruction for executing the exemplary manner described in the present disclosure.

The input/output (I/O) 230 may be used to input and/or output signals, data, information, etc. In some embodiments, the input/output (I/O) 230 may enable the user to interact with processing device 120. In some embodiments, the input/output (I/O) 230 may include an input device and an output device. An exemplary input device may include a keyboard, a mouse, a touch screen, a microphone, or any combination thereof. The exemplary output device may include a display device, a speaker, a printer, a projector, or any combination thereof. An exemplary display device may include a liquid crystal display (LCD), a light emitting diode (LED)-based display, a flat panel display, a curved surface display, a television device, a cathode ray tube, or any combination thereof.

The communication port 240 may be connected with a network (e.g., the network 150) to facilitate data communication. The communication port 240 may establish a connection between the processing device 120 and the imaging device 110, the terminal device 130, and/or the storage device 140. The connection may include a wired connection and a wireless connection. The wired connection may include, for example, cable, optical cable, telephone line, or any combination thereof. The wireless connection may include, for example, Bluetooth link, Wi-Fi™ link, WiMax™ link, WLAN link, ZigBee link, mobile network link (e.g., 3G, 4G, 5G), or any combination thereof. In some embodiments, the communication port 240 may be and/or include a standardized communication port, such as RS232, RS485, etc. In some embodiments, the communication port 240 may be a specially designed communication port. For example, the communication port 240 may be designed according to a digital imaging and medical communication (DICOM) protocol.

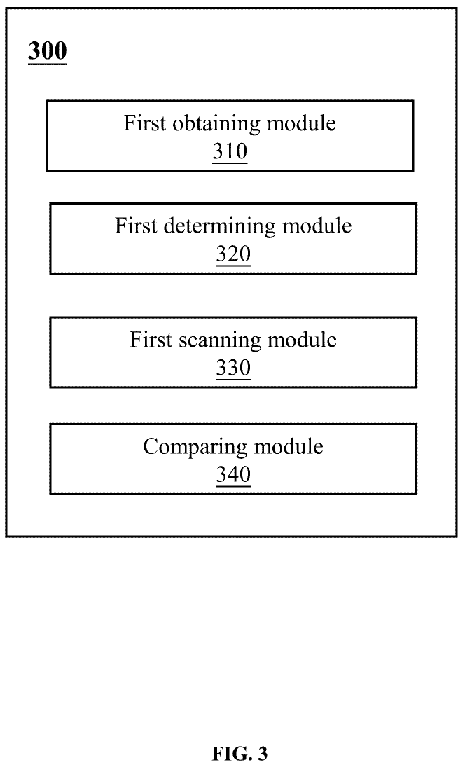

FIG. 3 is a block diagram illustrating an exemplary imaging system according to some embodiments of the present disclosure.

As illustrated in FIG. 3, in some embodiments, an imaging system 300 may include a first obtaining module 310, a first determining module 320, a first scanning module 330, and a comparing module 340. In some embodiments, the imaging system 300 may be integrated in the processing device 120 or the computing device 200.

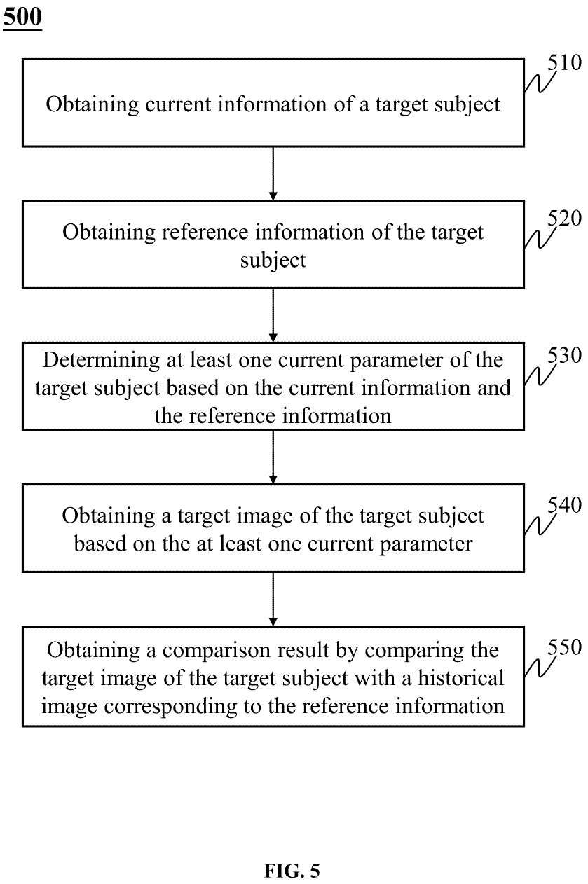

The first obtaining module 310 may be configured to obtain current information of a target subject. In some embodiments, the first obtaining module 310 may obtain the current information of the target subject from the storage device (e.g., the storage device 140). In some embodiments, the first obtaining module 310 may obtain the current information of the target subject from the terminal device (e.g., the terminal device 130) or the imaging device (e.g., the imaging device 110). In some embodiments, the first obtaining module 310 may obtain the current information of the target subject from the medical system. The current information may include information relating to current radiation attenuation of the target object. More descriptions for obtaining current information of a target subject may be found elsewhere in the present disclosure (e.g., operation 510 in FIG. 5 and descriptions thereof).

The first obtaining module 310 may also be configured to obtain reference information of the target subject. In some embodiments, the first obtaining module 310 may obtain the reference information from the personal database of the target subject. In some embodiments, the first obtaining module 310 may obtain the reference information of the target subject from the database based on an identification of the target subject. For example, the first obtaining module 310 may obtain the reference information of a patient from the database by matching the reference information based on an ID number, a phone number, or a clinic sequence number of the patient in the database of the patient. The reference information may include information relating to historical radiation attenuation of the target object. More descriptions for obtaining reference information of the target subject may be found elsewhere in the present disclosure (e.g., operation 520 in FIG. 5 and descriptions thereof).

The first determining module 320 may be configured to determine, based on the current information and the reference information, at least one current parameter of the target subject. In some embodiments, the first determining module 320 may obtain at least one historical parameter corresponding to the reference information, and determine, based on the current information, the reference information, and/or the at least one historical parameter, the at least one current parameter. In some embodiments, the first determining module 320 may obtain information variation between the current information and the reference information, and determine, based on the information variation and the at least one historical parameter, the at least one current parameter. In some embodiments, the first determining module 320 may obtain at least one initial parameter based on the at least one historical parameter, update the at least one initial parameter iteratively based on the information variation until a predicted image generated based on the at least one updated parameter and a historical image (corresponding to the at least one historical parameter) of the target subject satisfy a preset condition and determine the at least one updated parameter as the least one current parameter. In some embodiments, the first determining module 320 may determine the at least one current parameter automatically. In some embodiments, the first determining module 320 may determine the at least one current parameter automatically based on a user instruction. More descriptions for determining at least one current parameter of the target subject based on the current information and the reference information may be found elsewhere in the present disclosure (e.g., operation 530 in FIG. 5 and descriptions thereof).

The first scanning module 330 may be configured to obtain, based on the at least one current parameter, a target image of the target subject. In some embodiments, the first scanning module 330 may obtain scan data (e.g., an output of a detector) by scanning the target subject based on the current scanning parameter(s) and obtain the target image of the target subject by processing the scan data based on the historical processing parameter(s). In some embodiments, the first scanning module 330 may obtain scan data (e.g., an output of a detector) by scanning the target subject based on the historical scanning parameter(s) and obtain the target image of the target subject by processing the scan data based on the current processing parameter(s). In some embodiments, the first scanning module 330 may obtain scan data (e.g., an output of a detector) by scanning the target subject based on the current scanning parameter(s) or preset scanning parameter(s) and obtain the target image of the target subject by processing the scan data based on the current processing parameter(s) or preset processing parameter(s). More descriptions for obtaining a target image of the target subject based on the at least one current parameter may be found elsewhere in the present disclosure (e.g., operation 540 in FIG. 5 and descriptions thereof).

The comparing module 340 may be configured to obtain a comparison result by comparing the target image of the target subject with a historical image corresponding to the reference information. In some embodiments, the comparing module 340 may determine a corresponding base material image and a historical base material image by performing a base material decomposition on the target image and the historical image, respectively, and obtain a comparison result between the base material image and the historical base material image. More descriptions for obtaining a comparison result by comparing the target image of the target subject with a historical image corresponding to the reference information may be found elsewhere in the present disclosure (e.g., operation 550 in FIG. 5 and descriptions thereof).

More details about the first obtaining module 310, the first determining module 320, the first scanning module 330, and the comparing module 340 may be found in FIG. 5 and the related descriptions thereof, which is not limited herein.

It should be noted that the above description of the modules and the imaging system 300 is intended to be illustrative, and not to limit the scope of the present disclosure. It should be understood that, for persons having ordinary skills in the art, each module may be combined arbitrarily, or form a subsystem to be connected with other modules under the teachings of the present disclosure. However, those variations and modifications do not depart from the scope of the present disclosure.

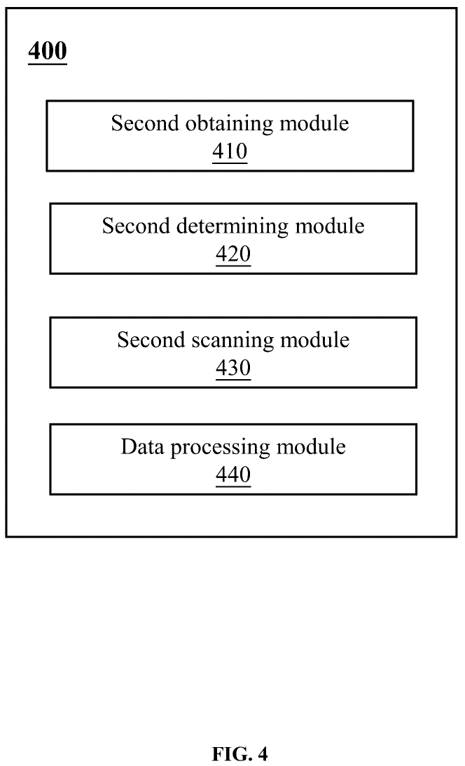

FIG. 4 is a block diagram illustrating an exemplary imaging system according to some embodiments of the present disclosure.

As illustrated in FIG. 4, in some embodiments, an imaging system 400 may include a second obtaining module 410, a second determining module 420, a second scanning module 430, and a data processing module 440. The second obtaining module 410 and the second determining module 420 may have a same or similar structure as the first obtaining module 310 and the first determining module 320 in the imaging system 300, respectively. In some embodiments, the imaging system 400 may be integrated in the processing device 120 or the computing device 200.

The second obtaining module 410 may be used to obtain related information of the target subject. In some embodiments, the second obtaining module 410 may be used to obtain current information of the target subject. In some embodiments, the second obtaining module 410 may be used to obtain reference information and at least one historical parameter of the target subject. The at least one historical parameter may include a historical scanning parameter and/or a historical processing parameter. In some embodiments, the related information may include a three-dimensional (3D) model, a body fat rate, etc., of the target subject. In some embodiments, the reference information, the historical scanning parameter, and the historical processing parameter may be obtained from a personal database of the target subject.

In some embodiments, the current information may include information relating to current radiation attenuation of the target object. In some embodiments, the reference information may include information relating to historical radiation attenuation of the target object.

In some embodiments, the current information may include information relating to at least one of a current body shape of the target subject, a current body fat rate of the target subject, or a current 3D model of the target object. In some embodiments, the reference information may include information relating to at least one of a reference body shape of the target subject, a reference body fat rate of the target subject, or a reference 3D model of the target object.

In some embodiments, the second obtaining module 410 may be used to obtain a historical image of the target subject from the personal database. In some embodiments, the target image may include a material decomposition image of the target subject. In some embodiments, the target image may have a same or a substantially similar confidence degree with the historical image, and the confidence degree may indicate an accuracy of the material decomposition image.

The second determining module 420 may be used to determine a current scanning parameter and/or a current processing parameter of the target subject.

In some embodiments, the second determining module 420 may be used to determine the current scanning parameter(s) of the target subject based on the current information, the reference information, and/or the at least one historical parameter. In some embodiments, the second determining module 420 may be used to obtain information variation by comparing the current information with the reference information of the target subject, and determine, based on the information variation and the at least one historical parameter, the current scanning parameter(s) of the target subject. The information variation may be related to radiation attenuation variation of the target subject. In some embodiments, the second determining module 420 may be used to convert the information variation into scanning parameter variation, and determine the current scanning parameter(s) of the target subject based on the scanning parameter variation and the historical scanning parameter(s).

In some embodiments, the second determining module 420 may be used to determine the current processing parameter(s) of the target subject based on the current information, the reference information, and/or the at least one historical parameter. In some embodiments, the second determining module 420 may be used to determine the current processing parameter(s) of the target subject based on the information variation and the historical processing parameter(s). In some embodiments, the second determining module 420 may be used to convert the information variation into processing parameter variation, and determine the current processing parameter(s) of the target subject based on the processing parameter variation and the historical processing parameter(s).

In some embodiments, the scanning parameter(s) may include at least one of a contrast agent type, a tube voltage, a tube current, a count of energy bins, a threshold value of each of the energy bins, an integral time corresponding to data in a single field of view, a rotating speed of a gantry, a thickness of a scan slice, etc. In some embodiments, the processing parameter(s) may include at least one of a weighting factor of the each of the energy bins, a noise reduction algorithm, a noise reduction level, a filter function, a reconstruction matrix, etc.

The second scanning module 430 may be used to scan the target subject. In some embodiments, the second scanning module 430 may be used to obtain an output of a detector by scanning the target subject based on the historical scanning parameter(s), the current scanning parameter(s), and/or preset scanning parameter(s).

The data processing module 440 may be used to process the output of the detector. In some embodiments, the data processing module 440 may be used to obtain the target image of the target subject by processing the output of the detector based on the historical scanning parameter(s), the current scanning parameter(s), and/or the preset scanning parameter(s).

In some embodiments, the data processing module 440 may be used to compare the target image with the historical image of the target subject and output a comparison result. In some embodiments, the comparison result may include a comparison result between a current base material image and a historical base material image of the target subject, and/or a comparison result between a current combined image and a historical combined image. The current combined image may be obtained by combining a plurality of current base materials based on a first set of weights, and the historical combined image may be obtained by combining a plurality of historical base materials based on a second set of weights. The first set of weights may be configured to make a numerical result of a non-target region of the current combined image be consistent with a numerical result of a non-target region of the historical combined image.

It should be noted that the above description of the modules and the imaging system 400 is intended to be illustrative, and not to limit the scope of the present disclosure. It should be understood, for persons having ordinary skills in the art, each module may be combined arbitrarily, or form a subsystem to be connected with other modules under the teachings of the present disclosure. However, those variations and modifications do not depart from the scope of the present disclosure.

FIG. 5 is a flowchart illustrating an exemplary imaging process according to some embodiments of the present disclosure.

In some embodiments, an imaging process 500 may be executed by the imaging system 100 (e.g., the processing device 120), the computing device 200 (e.g., the processor 210), or the imaging system 300. For example, the imaging method 300 may be implemented as a set of instructions or an application stored in a storage device (e.g., the storage device 140). The processing device 120 may execute the set of instructions or the application and may accordingly be directed to perform the imaging method 500.

In 510, current information of a target subject may be obtained. In some embodiments, operation 510 may be performed by the processing device 120 or the first obtaining module 310.

A target subject may be a subject to be scanned. In some embodiments, the target subject may include a patient to be scanned or a portion of the patient to be scanned.

In some embodiments, the current information may include a 3D model of the target subject. The 3D model may represent information such as organs or tissues included in a human body or a portion to be scanned, and/or a structure, density, volume, location, thickness, or the like, of the organs or tissues. In some embodiments, a processing device (e.g., the processing device 120 and/or the processor 210) may obtain the 3D model of the target subject through an image pick-up device (e.g., a depth camera), an infrared imager, a multi-angle 3D scanning (e.g., CT scanning, PET scanning, MR scanning, DR scanning, or the like), etc. For example, the processing device 120 may perform a thermal decomposition on a 3D image of the target subject obtained using the infrared imager to display information such as a distribution, location, size, or the like, of organs in the body of the target subject.

In some embodiments, the current information may include information relating to current radiation attenuation of the target object. In some embodiments, the current information may include an age, gender, a body shape (e.g., height and/or weight, a thickness of the target subject), a body fat rate, or the like, or any combination thereof. In some embodiments, the body fat rate may include a ratio of human body fat to the weight and/or fat in a certain organ/tissue of the human body to a weight of the organ/tissue. In some embodiments, the thickness may include a size (e.g., a straight-line distance between a certain point of a front chest to a back) between a front chest and back of the human body and/or a thickness of a portion of the target subject to be scanned (e.g., a thickness of an organ/tissue included in the portion to be scanned).

In some embodiments, the processing device (e.g., the processing device 120 and/or the processor 210) may obtain the current information of the target subject from the storage device (e.g., the storage device 140). In some embodiments, the processing device may obtain the current information of the target subject from the terminal device (e.g., the terminal device 130) or the imaging device (e.g., the imaging device 110). In some embodiments, the processing device may obtain the current information of the target subject from the medical system.

Current information may be related information of a current scanning of the target subject. For example, the current information of the target subject may include information such as a height, a weight, a thickness, a body fat rate, or the like, during the current scanning of the target subject. In some embodiments, the current related information may be input manually by a user (e.g., a typing input or a selection input of preset content through the terminal device 130 by a doctor) or obtained automatically by the system (e.g., obtained by scanning a clinic QR code of the target subject).

In 520, reference information of the target subject may be obtained. In some embodiments, operation 510 may be performed by the processing device 120 or the first obtaining module 310.

In some embodiments, the reference information may include information relating to historical radiation attenuation of the target object. The reference information may represent historical scanning information of the target subject. For example, the reference information may include information such as the 3D model, the body fat rate, or the like, during the last or previous scans of the target subject.

In some embodiments, the processing device (e.g., the processing device 120 and/or the processor 210) may obtain the reference information from the personal database of the target subject. In some embodiments, the processing device may obtain the reference information of the target subject from the database based on an identification of the target subject. For example, the processing device 120 may obtain the reference information of a patient from the database by matching the reference information based on an ID number, a phone number, or a clinic sequence number of the patient in the database of the patient.

In 530, at least one current parameter of the target subject may be determined based on the current information and the reference information. In some embodiments, operation 510 may be performed by the processing device 120 or the first determining module 320.

The at least one current parameter may be a parameter corresponding to a current scan (e.g., CT scan, MRI scan, PET scan, etc.) of the target subject.

In some embodiments, the processing device (e.g., the processing device 120 and/or the processor 210) may obtain at least one historical parameter corresponding to the reference information, and determine, based on the current information, the reference information, and/or the at least one historical parameter, the at least one current parameter.

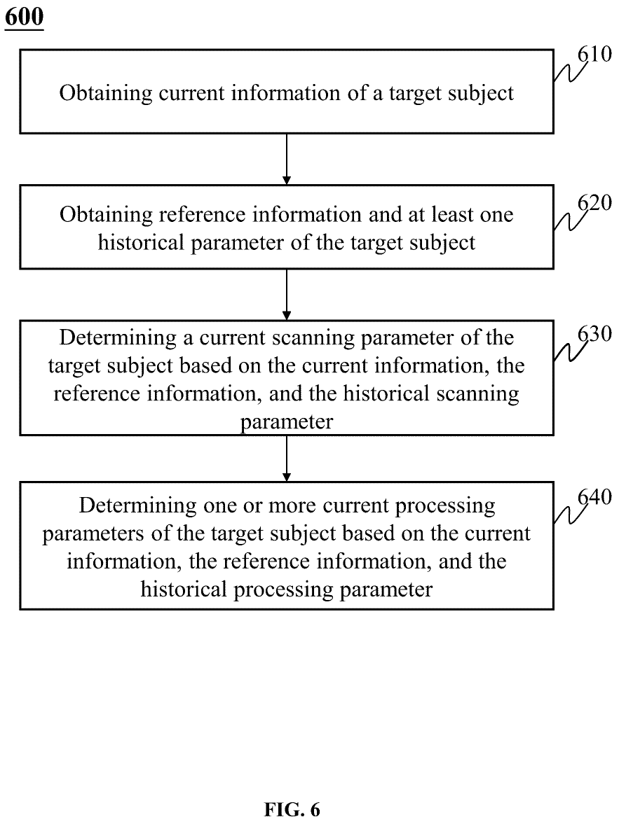

In some embodiments, the processing device may obtain information variation between the current information and the reference information, and determine, based on the information variation and the at least one historical parameter, the at least one current parameter. The information variation may be related to radiation attenuation variation of the target subject. More details may be found in FIG. 6 and the related descriptions thereof, which is not limited herein.

In some embodiments, the processing device may obtain at least one initial parameter based on the at least one historical parameter, update the at least one initial parameter iteratively based on the information variation until a predicted image generated based on the at least one updated parameter and a historical image (corresponding to the at least one historical parameter) of the target subject satisfy a preset condition and determine the at least one updated parameter as the least one current parameter.

In some embodiments, the preset condition may include that the predicted image has a same or a substantially similar confidence degree with the historical image, or a background region of the predicted image has at least one substantially same feature value as a background region of the historical image.

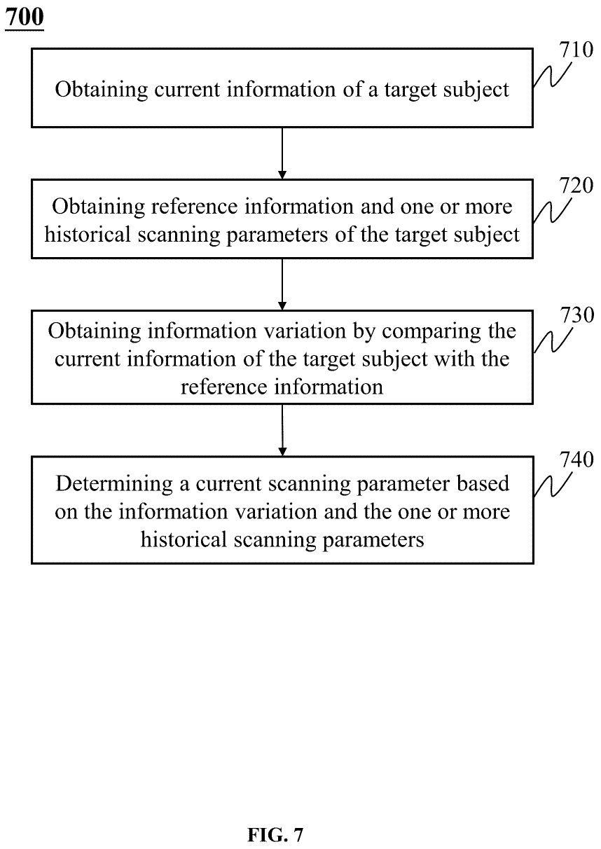

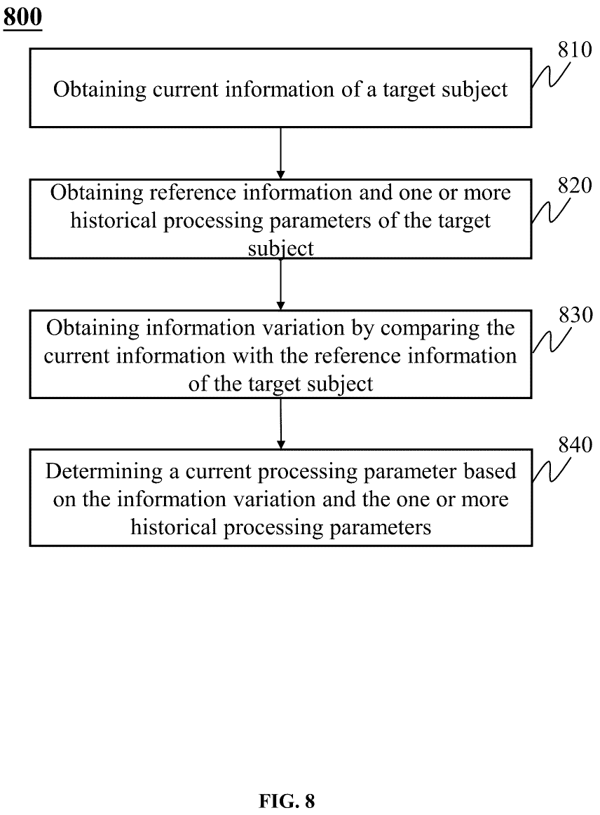

More details about determining the least one current parameter through iteratively updating may be found in FIG. 7, FIG. 8, and the related descriptions thereof, which is not limited herein.

In some embodiments, the at least one historical parameter may include at least one of a historical scanning parameter and a historical processing parameter. Correspondingly, the at least one current parameter may include at least one of a current scanning parameter and a current processing parameter.

A scanning parameter may represent a type and/or a corresponding numerical value of a parameter used in the scanning of the patient. A processing parameter may represent a parameter used during the processing of the scan data and/or image reconstruction performed based on the scan data.

In some embodiments, the processing device may determine the current scanning parameter(s) based on the current information, the reference information, and/or the historical scanning parameter. For example, the processing device 120 may compare the current information with the reference information of the target subject to obtain the information variation, and determine the current scanning parameter(s) of the target subject based on the information variation and the historical scanning parameter(s). More details may be found in FIG. 6, FIG. 7, and the related descriptions thereof, which is not limited herein.

In some embodiments, the processing device may determine, based on the current information, the reference information, and the historical processing parameter, the current processing parameter(s). For example, the processing device 120 may compare the current information with the reference information of the target subject to obtain the information variation, and determine the current processing parameter(s) of the target subject based on the information variation and the historical processing parameter(s). More details may be found in FIG. 6, FIG. 8, and the related descriptions thereof, which is not limited herein.

In some embodiments, the processing device may determine the at least one current parameter automatically. In some embodiments, the processing device may determine the at least one current parameter automatically based on a user instruction.

Merely by way of example, a user may select a parameter determination mode (e.g., automatic determination, manual input, or semi-automatic determination) from a preset mode of the imaging device 110 through an input device. In response to the user selecting the “automatic determination” mode, the imaging device 110 may generate an automatic determination instruction and transmit the automatic determination instruction to the processing device 120. In response to the automatic determination instruction, the processing device 120 may perform related operations of determining the at least one current parameter. For example, the processing device 120 may control the image pick-up device to obtain a 3D image of the target subject, determine the current information of the target subject based on the 3D image, and obtain the reference information from the personal database of the target subject. Furthermore, the processing device 120 may determine the at least one current parameter of the target subject based on the current information and the reference information.

In some embodiments, if historical clinic data (e.g., the reference information, the historical scanning parameter(s), and the historical processing parameter(s)) of the target subject is unable to be obtained, the processing device may determine preset parameter(s) based on the current information of the target subject. For example, for a first time of scan of the target subject, the target subject may have no reference information, no historical scanning parameter(s), and/or no historical processing parameter(s), the processing device 120 may determine one or more preset scanning parameters and/or one or more preset processing parameters based on the current information of the target subject. More details may be found in FIG. 6 and the related descriptions thereof, which is not limited herein.

In 540, a target image of the target subject may be obtained based on the at least one current parameter. In some embodiments, operation 540 may be performed by the processing device 120 or the first scanning module 330.

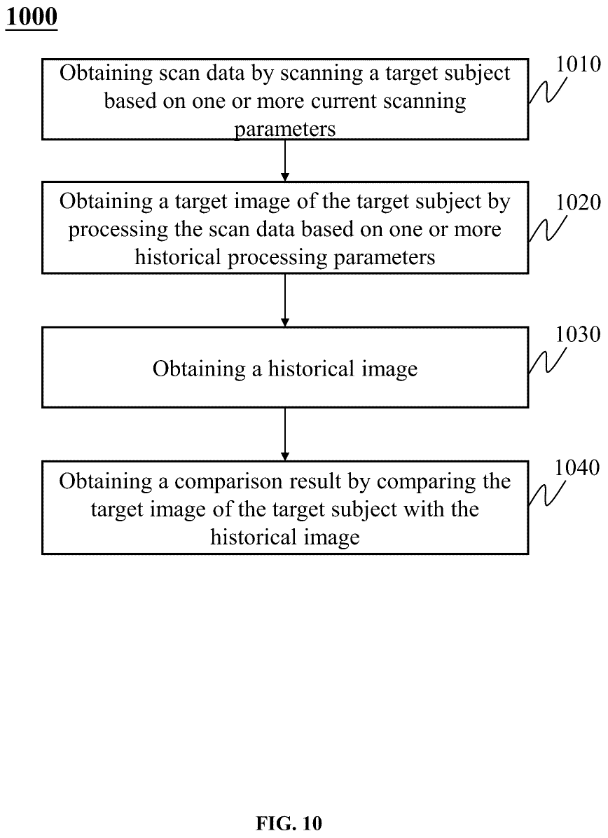

In some embodiments, the processing device (e.g., the processing device 120 and/or the processor 210) may obtain scan data (e.g., an output of a detector) by scanning the target subject based on the current scanning parameter(s) and obtain the target image of the target subject by processing the scan data based on the historical processing parameter(s). More details may be found in FIG. 10 and the related descriptions thereof, which is not limited herein.

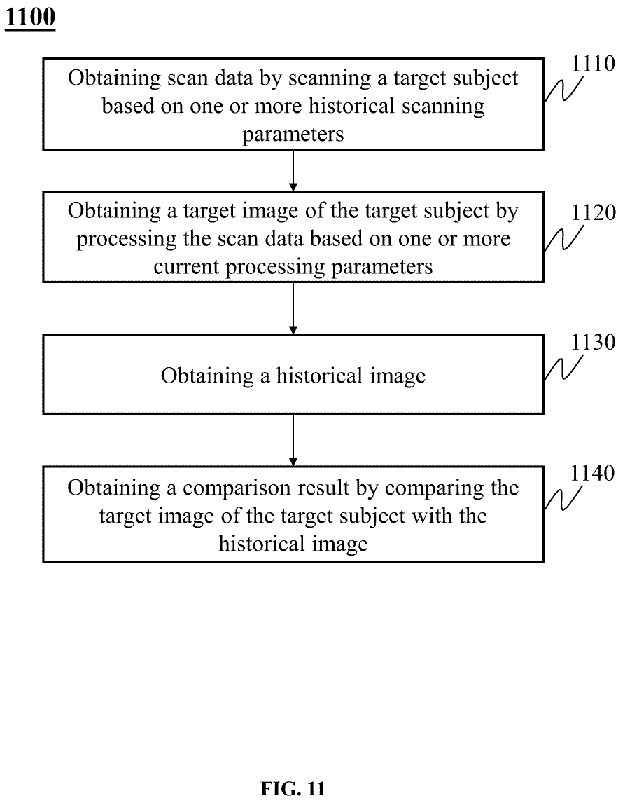

In some embodiments, the processing device may obtain scan data (e.g., an output of a detector) by scanning the target subject based on the historical scanning parameter(s) and obtain the target image of the target subject by processing the scan data based on the current processing parameter(s). More details may be found in FIG. 11 and the related descriptions thereof, which is not limited herein.

In some embodiments, the processing device may obtain scan data (e.g., an output of a detector) by scanning the target subject based on the current scanning parameter(s) or preset scanning parameter(s) and obtain the target image of the target subject by processing the scan data based on the current processing parameter(s) or preset processing parameter(s). More details may be found in FIG. 11 and the related descriptions thereof, which is not limited herein.

In some embodiments, the target image may include a material decomposition image of the target subject. In some embodiments, the target image may include a same or a substantially similar confidence degree with the historical image, and the confidence degree may indicate an accuracy of the material decomposition image.

In some embodiments, a background region of the target image may include at least one substantially same feature value as a background region of the historical image. The background region may be a region excluding a lesion region or a region excluding an organ or a tissue region. For example, the background region of the target image of the target subject may include a same feature value as the background region of the historical image.

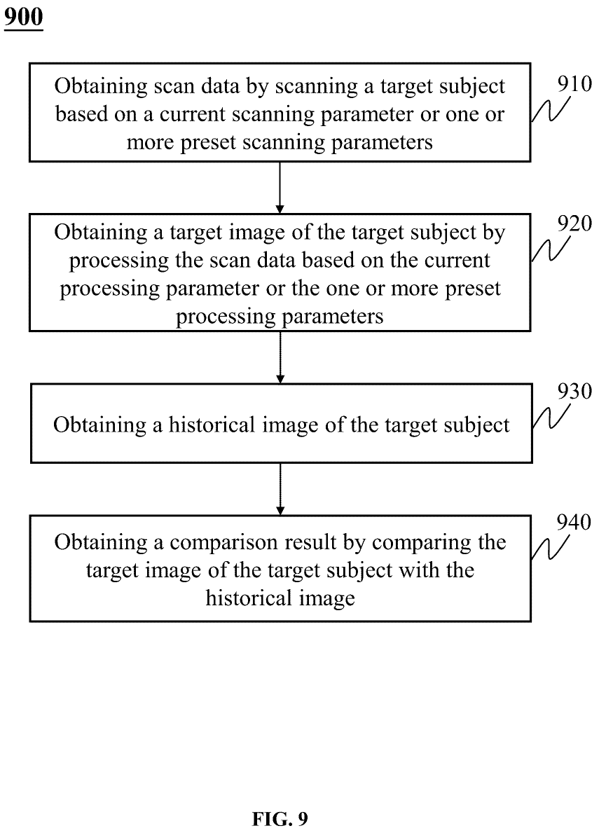

More details about obtaining the target image may be found in FIGS. 9-11 and the related descriptions thereof, which is not limited herein.

In 550, a comparison result may be obtained by comparing the target image of the target subject with a historical image corresponding to the reference information. In some embodiments, operation 510 may be performed by the processing device 120 or the comparing module 340.

The comparison result may represent a quantitative analysis result of material decomposition generated by comparing a current image (e.g., the target image) with the historical image. In some embodiments, the processing device (e.g., the processing device 120 and/or the processor 210) may determine a corresponding base material image and a historical base material image by performing a base material decomposition on the target image and the historical image, respectively, and obtain a comparison result between the base material image and the historical base material image. More details may be found in FIGS. 9-11 and the related descriptions thereof, which is not limited herein.

It should be noted that the above description regarding process 500 is merely provided for the purposes of illustration, and not intended to limit the scope of the present disclosure. For those skilled in the art, various modifications and variations may be made for the process 500 under the guidance of the present disclosure. However, these modifications and variations do not depart from the scope of the present disclosure.

FIG. 6 is a flowchart illustrating an exemplary process of determining at least one current parameter according to some embodiments of the present disclosure.

In some embodiments, a process 600 may be executed by the imaging system 100 (e.g., the processing device 120), the computing device 200 (e.g., the processor 210), or the imaging system 400. For example, the process 600 may be implemented as a set of instructions or an application stored in a storage device (e.g., the storage device 140). The processing device 120 may execute the set of instructions or the application and may accordingly be directed to perform the process 600.

In 610, current information of a target subject may be obtained. In some embodiments, operation 610 may be performed by the processing device 120 or the second obtaining module 410.

In some embodiments, the current information of the target subject may include at least one of a body shape (e.g., height, weight, and/or thickness), body fat rate, or the like. More details may be found in operation 510 and the related descriptions thereof.

In 620, reference information and at least one historical parameter of the target subject may be obtained. In some embodiments, the at least one historical parameter may include at least one of a historical scanning parameter or a historical processing parameter. In some embodiments, operation 620 may be performed by the processing device 120 or the second obtaining module 410.

Similar to the related descriptions of FIG. 5, a scanning parameter may represent a type and/or a corresponding numerical value of a parameter used in the scanning of the patient. In some embodiments, the scanning parameter may include a type of contrast agent, a tube voltage, a tube current, a count of energy bins, a threshold value of each of the energy bins, an integral time corresponding to data in a single field of view, a rotating speed of a gantry, a thickness of a scan slice, or the like, or any combination thereof.

A contrast agent (also referred to be a contrast media) may be a chemical product injected (or taken) into human tissues or organs to enhance an image observing effect. For example, species of the contrast agent may include Iodine (I), Aurum (Au), Rolling (Gd), Barium (Ba), etc.

Rays (e.g., X-rays) may be emitted from a ray tube during the medical imaging. The ray tube may include a cathode side (filament) and an anode side (target plane).

A tube voltage may be a voltage applied between the cathode side and the anode side of the ray tube to form a high voltage electric field, so that hot electrons emitted by the filament may bombard the target plane at a high speed under an acceleration of the high voltage electric field to excite the rays. A maximum photon energy of the rays generated by the ray tube may be equal to a maximum energy of a high-speed electron flow, and the maximum energy of the high-speed electron flow may depend on a peak value of the tube voltage. The maximum photon energy and a ray spectrum may be changed by changing the tube voltage.

A tube current may be current formed by the electrons generated by heating the filament moving to the anode at a high-speed under an action of two-stage high voltage electric field between the cathode and the anode. A product of the tube current and a time may determine an amount of rays (a count of emitted photons).

A photon counting detector (PCD) may count the photons with an energy higher than a preset threshold by setting different thresholds. Each incident photon may be counted based on a divided energy interval, so that raw CT data may include information in energy dimension. The divided energy interval may be referred as an energy bin, and the preset threshold may be referred as a threshold of an energy bin, which may indicate an interval size of each energy bin. A count of energy bins may be a count of energy intervals to be divided.

An integral time corresponding to data in a single field of view may be a time taken to collect a set of data (i.e., data corresponding to a single field of view) after the gantry rotating one circle during the CT imaging. For example, the gantry rotating one circle takes 1 second, and 9800 sets of data are collected, that is, 9800 field of views, thus, the integral time corresponding to data in a single field of view is 1/9800.

A rotating speed of a gantry may be a rotating speed of gantry of the imaging device (e.g., a CT imaging device).

A thickness of a scanning slice may be a thickness passed by the imaging device (e.g., a CT imaging device) during a scanning cycle. A slice thickness may be a length covered by a CT cross-section image in a direction perpendicular to an inspected subject (e.g., a target subject) or an examining table. The slice thickness may be related to a count of detector rows and/or detectors. The better the performance of the imaging device, the greater the count of detector rows, the thinner the thickness of the scanning slice, and the clearer the reconstructed image.

Similar to the related descriptions of FIG. 5, a processing parameter may represent a parameter used during the processing of the scan data and/or image reconstruction performed based on the scan data. In some embodiments, the processing parameter may include a weighting factor of the each of the energy bins, a noise reduction algorithm, a noise reduction level, a filter function, or a reconstruction matrix, or the like, or any combination thereof. The weighting factor of the each of the energy bins may represent a weight ratio of output data of the each energy bin of the detector when an output is processed by the detector. Parameters such as the weighting factor of the each of the energy bins, the noise reduction algorithm, the noise reduction level, the filter function, the reconstruction matrix, or the like, may have an effect on a quantitative result of material decomposition.

In some embodiments, the processing parameter may include a CT value, a window width, a window level, an artifact correction algorithm of an image, or the like, or any combination thereof. The CT value, the window width, the window level, the artifact correction algorithm of the image, or the like, may have an effect on an image quality of a material decomposition image.

A historical scanning parameter, a historical processing parameter, or the like, may represent historical scan information of the target subject corresponding to the reference information. For example, the historical scanning parameter may represent a scanning parameter used in the previous scans (e.g., the last scan, the last two times of scans, etc.) of the target subject. The historical processing parameter may represent a processing parameter used in previous post-processings of the target subject.

In some embodiments, the reference information, the historical scanning parameter(s), and the historical processing parameter(s) may be obtained from the personal database of the target subject. For example, the processing device 120 may establish a personal database for each patient. Related information such as the body fat rate, the weight, the 3D model of the patient, or the like, the scanning parameter(s) used during the scan of the patient, the post-processing parameter(s) used after the scan, the scanning image, the image examination result, or the like, that correspond to the current scan, may be archived and stored in the personal database corresponding to the patient.

In some embodiments, the processing device (e.g., the processing device 120 and the processor 210) may obtain the reference information, the historical scanning parameter(s), and/or the historical processing parameter(s) of the target subject from the database based on an identification of the target subject. For example, the processing device 120 may obtain the reference information, the historical scanning parameter(s), and/or the historical processing parameter(s) of the patient from a database by matching the reference information in the database of the patient based on an ID number, a phone number, or a clinic sequence number of the patient.

In 630, one or more current scanning parameters of the target subject may be determined based on the current information, the reference information, and the historical scanning parameter(s). In some embodiments, operation 630 may be performed by the processing device 120 or the second determining module 420.

In some embodiments, the processing device (e.g., the processing device 120 and/or the processor 210) may obtain information variation by comparing the current information and the reference information of the target subject. The information variation may be related to radiation attenuation variation of the target subject. The attenuation information may represent an attenuation degree relative to the rays. For example, an attenuation degree may be a product of an attenuation coefficient and an attenuation length.

In some embodiments, the processing device may determine the current scanning parameter(s) by determining whether to adjust the historical scanning parameter(s) based on the information variation. For example, in response to determining that the information variation is less than or equal to a first preset threshold or the information variation is within a first range, the processing device 120 may determine the historical scanning parameter(s) as the current scanning parameter(s) of the target subject directly; in response to determining that the information variation is greater than the first preset threshold or the information variation is within a second range, the processing device 120 may determine the current scanning parameter(s) by adjusting the historical scanning parameter(s).

In some embodiments, the processing device may transform the information variation into a scanning parameter variation, and the current scanning parameter(s) of the target subject may be determined based on the scanning parameter variation and the historical scanning parameter(s). More details may be found in FIG. 7 and related descriptions thereof, which is not limited herein.

In some embodiments, in response to determining that the information variation and/or the scanning parameter variation of the target subject are unable to be determined, the processing device may determine one or more preset scanning parameters based on the current information of the target subject. In some embodiments, the processing device may determine the preset scanning parameter(s) by adjusting value(s) of the preset scanning parameter(s) based on the current information of the target subject. In some embodiments, the processing device may determine the preset scanning parameter(s) by adjusting value(s) of the preset scanning parameter(s) based on a preset range. For example, if a first time of CT scan is performed on the patient, the target subject may have no historical clinic data, the preset scanning parameter(s) of the target subject may be determined by performing a linear adjustment (e.g., in response to determining that the weight of the target subject is greater than a weight corresponding to a standard value, the standard value may be increased; in response to determining that the weight of the target subject is less than the weight corresponding to the standard value, the standard value may be reduced; in response to determining that the weight of the target subject is equal to the weight corresponding to the standard value, the standard value may not be changed, or if a difference between the weight of the target subject and the weight corresponding to the standard value is greater than a preset differential value, the standard value may be increased, or if the difference between the weight of the target subject and the weight corresponding to the standard value is less than or equal to the preset differential value, the standard value may not be changed) on a preset standard value (e.g., a scanning parameter corresponding to a common body shape or a scanning parameter with a higher usage frequency) of a scanning parameter based on at least one of a scanning portion, a 3D model, a height, an age, gender, a weight of the patient, etc. As another example, parameters such as a Bin value, a Bin threshold, a tube current, or the like, may be changed due to a condition change of the contrast agent used in the current scan of the target subject, so that the scanning parameter variation of the target subject can not be determined directly, and the processing device 120 may determine the preset scanning parameter(s) of the target subject by performing a linear adjustment (e.g., in response to determining that the body fat rate of the target subject is greater than a body fat rate corresponding to a standard value, the standard value may be increased; in response to determining that the body fat rate of the target subject is less than the body fat rate corresponding to the standard value, the standard value may be reduced; in response to determining that the weight body fat rate of the target subject is equal to the body fat rate corresponding to the standard value, the standard value may not be changed, or if a difference between the body fat rate of the target subject and the body fat rate corresponding to the standard value is greater than a preset differential value, the standard value may be increased, or if the difference between the body fat rate of the target subject and the body fat rate corresponding to the standard value is less than or equal to the preset differential value, the standard value may not be changed) on a preset standard value (e.g., a scanning parameter corresponding to a common body fat rate or a 3D model) of a scanning parameter based on the type of the contrast agent, the 3D model, the body fat rate, etc. In some embodiments, standard values of scanning parameters corresponding to different types of information (e.g., the body shape, the body fat rate, the 3D mode, etc.) may be the same or different. In some embodiments, preset differential values of scanning parameter(s) corresponding to different types of information (e.g., the body shape, the body fat rate, the 3D mode, etc.) may be the same or different. For example, a preset differential value corresponding to the body fat rate that is used to determine whether the standard value is adjusted and a preset differential value corresponding to the body shape that is used to determine whether the standard value is adjusted may be different.

In 640, one or more current processing parameters of the target subject may be determined based on the current information, the reference information, and the historical processing parameter(s). In some embodiments, operation 640 may be performed by the processing device 120 or the second determining module 420.

In some embodiments, the processing device (e.g., the processing device 120 and/or the processor 210) may determine the current processing parameter(s) of the target subject based on the information variation and the historical processing parameter(s). In some embodiments, the processing device may determine the current processing parameter(s) by determining whether to adjust the historical processing parameter(s) based on the information variation. For example, in response to determining that the information variation is less than or equal to a second preset threshold or the information variation is within a third range, the processing device 120 may determine the historical processing parameter(s) as the current processing parameter(s) of the target subject directly; in response to determining that the information variation is greater than the second preset threshold or the information variation is within a fourth range, the processing device 120 may determine the current processing parameter(s) by adjusting the historical processing parameter(s).

In some embodiments, in response to determining that the information variation exceeds a preset threshold (e.g., the first preset threshold and the second preset threshold) or the information variation is within a preset range (e.g., the second range and the fourth range), the processing device may determine the current scanning parameter(s) and/or the current processing parameter(s) of the target subject by adjusting the historical scanning parameter(s) and the historical processing parameter(s) simultaneously or merely adjusting one of the historical scanning parameter(s) and the historical processing parameter(s). For example, the processing device 120 may determine the historical scanning parameter(s) of the target subject as the current scanning parameter(s), and determine the current processing parameter(s) by adjusting the historical processing parameter(s) of the target subject based on the information variation. As another example, the processing device 120 may determine the current scanning parameter(s) by adjusting the historical scanning parameter(s) of the target subject based on the information variation, and determine the historical processing parameter(s) of the target subject as the current processing parameter(s). That is, the current scanning parameter(s) may be different from the historical scanning parameter(s), and the current processing parameter(s) may be the same as the historical processing parameter(s). As a further example, the processing device 120 may determine the current scanning parameter(s) and the current processing parameter(s) of the target subject by adjusting the historical scanning parameter(s) and the historical processing parameter(s) based on the information variation of the target subject. That is, the current scanning parameter(s) and the current processing parameter(s) may be different from the historical scanning parameter(s) and the historical processing parameter(s).

In response to determining that the information variation is greater than the preset threshold or the information variation is within the second range or the fourth range, a variation of current attenuation information of the patient may be greater in comparison with attenuation information obtained from last scan. The historical scanning parameter(s) and/or the historical processing parameter(s) may be adjusted to ensure that a scanning result of the patient obtained before and/or after a scan includes quantitative comparison, so that an image obtained based on the current scan may satisfy clinic requirements.

In some embodiments, the processing device may obtain an output of a detector by scanning the target subject based on the current scanning parameter(s), and a target image of the target subject may be obtained by processing the output of the detector based on the current processing parameter(s). In some embodiments, the processing device may output a comparison result by comparing the target image of the target subject with a historical image. More details may be found in FIGS. 9-11 and related descriptions thereof, which are not limited herein.

It should be noted that the above description regarding process 600 is merely provided for the purposes of illustration, and not intended to limit the scope of the present disclosure. For those skilled in the art, various modifications and variations may be made for the process 600 under the guidance of the present disclosure. For example, operation 640 may be executed before operation 630, or operation 630 and operation 640 may be executed simultaneously, or merely one of operation 630 and operation 640 may be executed. However, these modifications and variations do not depart from the scope of the present disclosure.

FIG. 7 is a flowchart illustrating an exemplary process of determining a current scanning parameter according to some embodiments of the present disclosure.

In some embodiments, a process 700 may be executed by the imaging system 100 (e.g., the processing device 120), the computing device 200 (e.g., the processor 210), or the imaging system 400. For example, the process 700 may be implemented as a set of instructions or an application stored in a storage device (e.g., the storage device 140). The processing device 120 may execute the set of instructions or the application and may accordingly be directed to perform the process 700.

In 710, current information of a target subject may be obtained. In 720, reference information and one or more historical scanning parameter(s) of the target subject may be obtained. Operations 710 and 720 may be similar to operations 610 and 620 of the process 600, respectively. More details may be found in FIG. 6 and related descriptions thereof, which is not limited herein.

In 730, information variation may be obtained by comparing the current information of the target subject with the reference information. In some embodiments, operation 730 may be performed by the processing device 120 or the second determining module 420.

Information variation may represent a change of attenuation degree caused by a change of related information of the target subject.

In some embodiments, the information variation may be determined based on an attenuation coefficient and an attenuation length. Different materials (e.g., different tissues, organs, potions of a human body, different types of contrast agent, or the like) may correspond to different attenuation coefficients of X-rays with different energies. The attenuation length may depend on a length of an attenuation model (e.g., a 3D model of a target subject) in an X-ray optical path, which also may be considered as a thickness (e.g., a thickness of the target subject) of the patient or a portion of the patient to be scanned.

In some embodiments, the processing device (e.g., the processing device 120 and/or the processor 210) may determine the information variation based on an attenuation coefficient and an attenuation length of the current information of the target subject and an attenuation coefficient and an attenuation length of the reference information. In some embodiments, the processing device may determine the information variation based on a difference between the attenuation length of the current information and the attenuation length of the reference information and a difference between an average attenuation coefficient of the current information and an average attenuation coefficient of the reference information.

Merely by way of example, the information variation may be denoted as ΔB, and ΔB=(L+ΔL)×(α+Δα)−L×α, wherein L refers to an attenuation length of reference information B1 of the target subject, ΔL refers to a difference between an attenuation length of current information B2 of the target subject and an attenuation length of the reference information B1, α refers to an average attenuation coefficient corresponding to B1, and Δα refers to a difference between an average attenuation coefficient of B1 and an average attenuation coefficient of B2.

In some embodiments, the processing device may determine the attenuation coefficient and/or the attenuation length based on at least one of age, gender, height, weight, body fat rate, a 3D model of the target subject, or the like. For example, the processing device 120 may extract the attenuation length L (corresponding to a scan position and a scanned portion of the patient to be scanned) exposed in the X-ray optical path, and/or, extract the average attenuation coefficient α of the human tissues (corresponding to a scan position and a scanned portion of the patient to be scanned) exposed in the X-ray optical path based on the age, gender, height, weight, body fat rate, and/or the 3D model corresponding to the reference information B1 of the target subject. As another example, the processing device 120 may determine a corresponding attenuation length and/or a corresponding attenuation coefficient based on the age, gender, height, and weight corresponding to the current information B2 of the target subject, by simplifying the target subject as a 3D barrel model or bringing the target subject into a 3D standard model. As a further example, the processing device 120 may determine a corresponding attenuation length and/or a corresponding attenuation coefficient based on a 3D model corresponding to the current information B2 of the target subject, by simplifying the target subject as a 3D barrel model or bringing the target subject into a 3D standard model.

It should be understood that the above description regarding a determination of information variation is merely provided as an example. In some embodiments, the information variation may be determined through other manners, which is not limited herein.

In 740, the current scanning parameter(s) may be determined based on the information variation and one or more historical scanning parameters. In some embodiments, operation 740 may be performed by the processing device 120 or the second determining module 420.

Combined with the illustrations mentioned above, in some embodiments, in response to determining that the information variation is less than or equal to the first preset threshold or the information variation is within the first range, the processing device may determine the historical scanning parameter(s) of the target subject as the current scanning parameter(s). The preset threshold may be any suitable numerical value, which is not limited herein.

In some embodiments, in response to determining that the information variation is greater than the first preset threshold or the information variation is within the second range, the processing device may transform the information variation into a scanning parameter variation, and the current scanning parameter(s) of the target subject may be determined based on the scanning parameter variation and the historical scanning parameter(s). The scanning parameter variation may represent a value required to be adjusted corresponding to each scanning parameter or a target value to be reached.

In some embodiments, the scanning parameter variation may include at least one of a type of contrast agent, a tube voltage, a tube current, a count of energy bins, a threshold value of each of the energy bins, an integral time corresponding to data in a single field of view, a rotating speed of a gantry, or a thickness of a scan slice, or the like, or any combination thereof.

In some embodiments, the processing device may determine the scanning parameter variation based on a usage condition (e.g., whether uses contrast agent, a type of used contrast gent, or the like) of the contrast agent. For example, a setting of the tube voltage, the count of energy bins, and the threshold value of each of the energy bins (i.e., a historical value is used) may be kept constant under a scanning scenario that the usage condition of the contrast agent is not changed. A scanning parameter variation ΔS may include at least one of a tube current variation ΔmA, an integral time variation corresponding to data in a single field of view Δt, a thickness variation of a scan slice, or the like, or any combination thereof. As another example, an optimal scanning parameter adjustment combination may be determined as the scanning parameter variation ΔS by performing a calculation based on a preset parameter adjustment priority through an iterative manner under a scanning scenario that the usage condition of the contrast agent is required to be changed.

In some embodiments, the processing device may determine the scanning parameter variation by ensuring that an output characteristic of each energy bin of a detector is comparable to that in a previous scanning. In some embodiments, the processing device may obtain a corresponding relationship between the attenuation information and the scanning parameter(s) under the premise of ensuring that outputs of different energy bins of a detector are consistent. For example, the processing device 120 may obtain the corresponding relationship (e.g., a corresponding relationship between a thickness of a 3D model and a tube current) between the attenuation information and the scanning parameter(s) by performing a simulation scanning test on a plurality of 3D models with different attenuation capabilities of a human body under the premise of ensuring that the outputs of different energy bins of the detector are consistent. In some embodiments, the processing device may transform the information variation into the scanning parameter variation based on the corresponding relationship between the attenuation information and the scanning parameter(s).

In some embodiments, the processing device may transform the information variation into the scanning parameter variation through an iterative computation. For example, the processing device may determine the information variation of the target subject as an input, and determine the scanning parameter variation by performing the iterative computations on a set of scanning parameters.

In some embodiments, the processing device may determine the current scanning parameter(s) of the target subject by adjusting the historical scanning parameter(s) of the target subject based on the scanning parameter variation. For example, the processing device 120 may determine the current scanning parameter(s) of the target subject by adjusting a value of a corresponding type of scanning parameter based on the scanning parameter variation.

In some embodiments, the processing device may update the historical scanning parameter(s) iteratively based on the information variation until a predicted image generated based on the updated historical scanning parameter(s) and the historical image satisfy a preset condition, and determine the updated historical scanning parameter(s) as the current scanning parameter(s) of the target subject.

The predicted image may be a scanning image obtained through a prediction based on the at least one updated parameter. For example, the predicted image may include a scanning image of the target subject obtained through a simulation based on the updated historical scanning parameter(s) and the updated historical processing parameter(s), or a scanning image of the target subject obtained through a simulation based on un-updated historical scanning parameter(s) and the updated historical processing parameter(s), or a scanning image of the target subject obtained through a simulation based on the updated historical scanning parameter(s) and un-updated historical processing parameter(s).

Combined with the illustrations mentioned above, in some embodiments, the preset condition may include that the predicted image has a same or a substantially similar confidence degree (e.g., a differential value is less than a preset value) with the historical image. The confidence degree may represent an accuracy a quantitative result shown in the material decomposition image. In some embodiments, the preset condition may include that a background region of the predicted image has at least one same or substantially same (e.g., a difference is less than a preset value) feature value as a background region of the historical image. The feature value may include a value representing information of the background region, such as a pixel value, a CT value of the background region, or the like.

Merely by way of example, the processing device 120 may obtain one or more preliminary scanning parameters by updating the historical scanning parameter(s) based on the information variation. The processing device 120 may determine a preliminary predicted image of the target subject based on the preliminary scanning parameter(s), and determine whether confidence degrees of the preliminary predicted image and the historical image are the same. In response to determining that the confidence degrees are the same, the processing device 120 may determine the preliminary scanning parameter(s) as the current scanning parameter(s). In response to determining that the confidence degrees are not the same, the processing device 120 may obtain updated scanning parameter(s) by updating the preliminary scanning parameter(s) based on the information variation, and determine whether a confidence degree of a predicted image corresponding to the updated scanning parameter is the same as a confidence degree of the historical image. In response to determining that the confidence degrees are not the same, the processing device 120 may update the updated scanning parameter(s) based on the information variation and repeat the process mentioned above, until the confidence degree of the predicted image corresponding to the updated scanning parameter is the same as the confidence degree of the historical image, and the processing device 120 may determine the updated scanning parameter(s) as the current scanning parameter(s).

In some embodiments, the processing device may obtain the scan data (e.g., an output of a detector) and/or the target image of the target subject by scanning the target subject based on the current scanning parameter(s).

It should be noted that the above description regarding process 700 is merely provided for the purposes of illustration, and not intended to limit the scope of the present disclosure. For those skilled in the art, various modifications and variations may be made for the process 700 under the guidance of the present disclosure. However, these modifications and variations do not depart from the scope of the present disclosure.

FIG. 8 is a flowchart illustrating an exemplary process of determining a current processing parameter according to some embodiments of the present disclosure. In some embodiments, a process 800 may be executed by the imaging system 100 (e.g., the processing device 120), the computing device 200 (e.g., the processor 210), or the imaging system 400. For example, the process 800 may be implemented as a set of instructions or an application stored in a storage device (e.g., the storage device 140). The processing device 120 may execute the set of instructions or the application and may accordingly be directed to perform the process 800.

In 810, current information of a target subject may be obtained. In 820, reference information and one or more historical processing parameters of the target subject may be obtained. Operations 810 and 820 may be similar to operations 610 and 620 of the process 600, respectively. More details may be found in FIG. 6 and related descriptions thereof. In 830, information variation may be obtained by comparing the current information with the reference information of the target subject. Operation 830 may be similar to operation 730 of the process 700. More details may be found in FIG. 7 and related descriptions thereof, which is not limited herein.

In 840, the current processing parameter(s) may be determined based on the information variation and one or more historical processing parameters. In some embodiments, operation 840 may be performed by the processing device 120 or the second determining module 420.

Combined with the illustrations mentioned above, in some embodiments, in response to determining that the information variation is less than or equal to the second preset threshold or the information variation is within the third range, the processing device (e.g., the processing device 120 and the processor 210) may determine the historical processing parameter(s) of the target subject as the current processing parameter(s). That is, the current processing parameter(s) may be the same as the historical processing parameter(s). In some embodiments, the first preset threshold and the second preset threshold may be the same or different. In some embodiments, the third range and the first range may be the same or different.

In some embodiments, in response to determining that the information variation is greater than the second preset threshold or the information variation is within the fourth range, the processing device may determine the current processing parameter(s) of the target subject by adjusting the historical processing parameter(s). In some embodiments, the second range and the fourth range may be the same or different. In some embodiments, the processing device may transform the information variation into a processing parameter variation, and determine the current processing parameter(s) of the target subject based on the processing parameter variation and the historical processing parameter(s).

In some embodiments, combined with the illustrations mentioned above, the current processing parameter(s) may include at least one of a weighting factor of the each of the energy bins, a noise reduction algorithm, a noise reduction level, a filter function, a reconstruction matrix, or the like.

In some embodiments, the processing device may determine the current processing parameter(s) based on an output characteristic of the each of the energy bins of the detector. For example, if a signal-to-noise ratio of an output of an energy bin of the detector corresponding to the current scan is worse than a signal-to-noise ratio of an output of the energy bin during the last scan corresponding to the historical processing parameter(s), a weighting factor of the energy bin may be reduced.

In some embodiments, in response to determining that the information variation of the target subject is unable to be determined, the processing device may determine one or more preset processing parameters based on the current information of the target subject and/or the output of the detector during the current scan. For example, the processing device 120 may determine the preset processing parameter(s) of the patient based on the output of the detector during the current scan for a first CT scan performed on the patient.

In some embodiments, the processing device may determine the preset processing parameter(s) by adjusting value(s) of the preset processing parameter(s) based on the current information of the target subject. In some embodiments, the processing device may determine the preset processing parameter(s) by adjusting value(s) of the preset processing parameter(s) based on a preset range. For example, for a first time of CT scan performed on the patient, the processing device 120 mat determine the preset processing parameter(s) of the target subject by performing a linear adjustment (e.g., in response to determining that the weight of the target subject is greater than a weight corresponding to a standard value, the standard value may be increased; in response to determining that the weight of the target subject is less than the weight corresponding to the standard value, the standard value may be reduced; in response to determining that the weight of the target subject is equal to the weight corresponding to the standard value, the standard value may not be changed) on a preset standard value (e.g., a processing parameter corresponding to a common body shape, body fat rate, or a 3D model, or a processing parameter with a higher usage frequency) of a processing parameter based on at least one of a scanning portion, a 3D model, a body shape, a body fat rate, a thickness of the target subject, etc.

In some embodiments, the processing device may update the historical processing parameter(s) iteratively based on the information variation until a predicted image generated based on the updated historical processing parameter(s) and the historical image satisfy a preset condition, and determine the updated historical processing parameter(s) as the current processing parameter(s) of the target subject.

Merely by way of example, the processing device 120 may obtain one or more preliminary processing parameters by updating the historical processing parameter(s) based on the information variation. The processing device 120 may determine a preliminary predicted image of the target subject based on the preliminary processing parameter(s), and determine whether pixel values of a background region of the preliminary predicted image and pixel values of a background region of the historical image are the same. In response to determining that the pixel values are the same, the processing device 120 may determine the preliminary processing parameter(s) as the current processing parameter(s). In response to determining that the pixel values are not the same, the processing device 120 may obtain updated processing parameter(s) by updating the preliminary processing parameter(s) based on the information variation, and determine whether pixel values of the background region of a predicted image corresponding to the updated processing parameter(s) are the same as pixel values of the background region of the historical image. In response to determining that the pixel values are not the same, the processing device 120 may update the updated processing parameter(s) based on the information variation and repeat the process mentioned above, until the pixel values of the background region of a predicted image corresponding to the updated processing parameter(s) are the same or substantially the same as the pixel values of the background region of the historical image, and the processing device 120 may determine the updated processing parameter(s) as the current processing parameter(s).

In some embodiments, the processing device may obtain the target image of the target subject by processing the scan data (e.g., an output of a detector) of the target subject based on the current processing parameter(s).

Factors having an effect on an accuracy of a material quantitative decomposition result may include a Bin value of the scanning parameter(s) and a signal-to-noise ratio of data of each Bin. Other types of scanning parameters and processing parameters may have an effect on the accuracy of the material quantitative decomposition result through an effect on the Bin value and the signal-to-noise ratio of the data of each Bin. For example, a combination of a Bin threshold, a tube voltage, and a tube current may be used to determine a count of photons distributed in each Bin, thereby having an effect on the signal-to-noise ratio. The signal-to-noise ratio may be changed with the noise reduction algorithm, the noise reduction level, and/or the filter function. Therefore, the current scanning parameter(s) and/or the current processing parameter(s) may be determined under a condition that a signal-to-noise ratio of the each Bin data is comparable to a previous scanning result, so that an accuracy of a material quantitative decomposition result can be improved.

It should be noted that the above description regarding process 800 is merely provided for the purposes of illustration, and not intended to limit the scope of the present disclosure. For those skilled in the art, various modifications and variations may be made for process 800 under the guidance of the present disclosure. However, these modifications and variations do not depart from the scope of the present disclosure.

FIG. 9 is a flowchart illustrating an exemplary imaging process according to some embodiments of the present disclosure.

As illustrated in FIG. 9, in some embodiments, a target image may be determined based on one or more current scanning parameter(s) or one or more preset scanning parameter(s), and one or more current processing parameter(s) or one or more preset processing parameter(s) of a target subject, and a comparison result may be determined based on the target image and a historical image. In some embodiments, the scanning parameter(s) (or the processing parameter(s)) corresponding to the current scan of the target subject may be different from the historical scanning parameter(s) (or the historical processing parameter(s)). That is, if the information variation is greater than the preset threshold, the current scanning parameter(s) (or the current processing parameter(s)) of the target subject may be obtained by adjusting the historical scanning parameter(s) (or the historical processing parameter(s)) based on the information variation.

In some embodiments, an imaging method 900 may be executed by the imaging system 100 (e.g., the processing device 120), the computing device 200 (e.g., the processor 210), or the imaging system 400. For example, the imaging method 900 may be implemented as a set of instructions or an application stored in a storage device (e.g., the storage device 140). The processing device 120 may execute the set of instructions or the application and may accordingly be directed to perform the imaging method 900.

In 910, scan data may be obtained by scanning the target subject based on the current scanning parameter(s) or one or more preset scanning parameter(s). In some embodiments, operation 910 may be performed by the processing device 120 or the second scanning module 430.

In some embodiments, the scan data may include an output (e.g., a photon counting value) of a detector. In some embodiments, the processing device may perform a parameter setting based on the current scanning parameter(s) or the preset scanning parameter(s) to scan the target subject, and the output of the detector of the imaging device (e.g., the imaging device 110) may be obtained. In some embodiments, the scanning parameter(s) may be set by the system automatically, or the scanning parameter(s) may be adjusted by the user manually. For example, the system 100 may set value(s) of corresponding parameter(s) to be matched to the current scanning parameter(s) or the preset scanning parameter(s) automatically, or an medical staff may input value(s) of corresponding scanning parameter(s) manually through the terminal device 130 or an input device of the imaging device 110.

In some embodiments, the output of the detector may include a photon counting value of each energy bin.

In 920, a target image of the target subject may be obtained by processing the scan data based on the current processing parameter(s) or the preset processing parameter(s). In some embodiments, operation 920 may be performed by the processing device 120 or the data processing module 440.

In some embodiments, the processing device may obtain the target image of the target subject by processing the output of the detector based on one or more of the current processing parameter(s) or the preset processing parameter(s). For example, the processing device 120 may obtain the target image of the target subject by performing a weighting computation on an output of each energy bin of the detector based on a determined or preset weighting factor of each energy bin and a reconstruction computation based on a reconstruction matrix. As another example, the processing device 120 may obtain the target image of the target subject by performing the weighting computation on the output of the energy bin(s) of the detector based on the determined or preset weighting factor(s) of the energy bin(s), a noise reduction computation based on determined noise reduction algorithm(s) and a noise reduction level, and/or a computation based on filter function(s) and reconstruction matrix(es).

Combined with the illustrations mentioned above, the target image may include a material decomposition image of the target subject. The material decomposition image (also referred to as a base material image) may be an image obtained by performing a base material decomposition on the output of the detector based on a difference between material attenuation coefficients under different energies. For example, a “water-iodine” image, a “water-calcium” image, a “water-iodine-calcium” image, a “water-calcium-metal” image, a fat image, a uric acid image, or the like, may be determined through decomposition.

As illustrated above, the target image of the target subject may include a same or a substantially similar confidence degree with the historical image. In some embodiments, a target material decomposition image of the target subject may include the same or the substantially similar confidence degree with a historical material decomposition image. For example, a background region of the target image may include a same or a substantially similar image quality with a background region of the historical image, such that the target image includes the same confidence level with the historical image, and an accuracy of a quantitative result of the material decomposition of the target image is the same as an accuracy of a quantitative result of the material decomposition of the historical image.

In 930, a historical image of the target subject may be obtained. In some embodiments, operation 930 may be performed by the processing device 120 or the second obtaining module 410.

In some embodiments, the historical image may be obtained from a personal database of the target subject. In some embodiments, the historical image may include a reconstruction image obtained from a previous scan (e.g., the last scan) of the target subject. In some embodiments, the historical image may include a plurality of reconstruction images obtained from a plurality of previous scans of the target subject.

In 940, a comparison result may be obtained by comparing the target image of the target subject with the historical image. In some embodiments, operation 940 may be performed by the processing device 120 or the data processing module 440.

A comparison result may represent a quantitative analysis result of material decomposition generated by comparing a current image with the historical image. In some embodiments, the comparison result may include at least one of a comparison result between a current base material image and a historical base material image and a comparison result between a current combined image and a historical combined image. The current combined image may be obtained by combining a plurality of current base materials based on a first set of weights, and the historical combined image may be obtained by combining a plurality of historical base materials based on a second set of weights. The first set of weights may be configured to make a numerical result of a non-target region of the current combined image be consistent with a numerical result of a non-target region of the historical combined image.

In some embodiments, the processing device may determine the base material image by performing the base material decomposition on the target image and the historical image and obtain the comparison result between the current base material image and a historical base material image. In some embodiments, the processing device may obtain the historical base material image of the target subject from the database. In some embodiments, the comparison result of the base material images may include a numerical value (e.g., a content value) of the current base material image and a numerical value (e.g., a content value) of the historical base material image, and/or a difference between the numerical values.

A first set of weights may represent a weighting factor of each base material corresponding to the current base material image. Correspondingly, a second set of weights may represent a weighting factor of each base material corresponding to the historical base material image. In some embodiments, the first set of weights and the second set of weights may be the same or different.

In some embodiments, the processing device may combine two or more base material images based on the first set of weights to obtain a current combined image. In some embodiments, the processing device may combine two or more historical base material images based on the second set of weights to obtain a historical combined image. In some embodiments, a comparison result between the current combined image and the historical combined image may include an image, a numerical value, a difference, or the like, or any combination thereof. For example, the processing device 120 may output and display the current combined image and the historical combined image to a medical staff, or output and display the difference between the current combined image and the historical combined image to the medical staff.

A non-target region may be a tissue region that is of no interest or of no significant interest, such as a normal tissue region, a background region, or the like. In some embodiments, keeping a numerical result of a non-target region of the current combined image to be consistent with a numerical result of a non-target region of the historical combined image may indicate that a CT value or other equivalent numerical values of a non-target region of the current combined image is consistent with a CT value or other equivalent numerical values of a non-target region of the historical combined image.

The numerical result of the non-target region of the current combined image may be kept to be consistent with the numerical result of the non-target region of the historical combined image, so that a change of a target region (e.g., a lesion region) of the patient may be highlighted, thereby improving the diagnosis efficiency and the accuracy of the diagnosis result.

It should be noted that the above description regarding process 900 is merely provided for the purposes of illustration, and not intended to limit the scope of the present disclosure. For those skilled in the art, various modifications and variations may be made for the process 900 under the guidance of the present disclosure. However, these modifications and variations do not depart from the scope of the present disclosure.

FIG. 10 is a flowchart illustrating an exemplary imaging process according to some embodiments of the present disclosure. In some embodiments, an imaging process 1000 may be executed by the imaging system 100 (e.g., the processing device 120), the computing device 200 (e.g., the processor 210), or the imaging system 400. For example, the imaging method 1000 may be implemented as a set of instructions or an application stored in a storage device (e.g., the storage device 140). The processing device 120 may execute the set of instructions or the application and may accordingly be directed to perform the imaging method 1000.

In 1010, scan data may be obtained by scanning a target subject based on one or more current scanning parameters. In some embodiments, operation 1010 may be performed by the processing device 120 or the second scanning module 430.

In some embodiments, the processing device may scan the target subject by performing a parameter setting based on the current scanning parameter(s) and obtain an output of a detector of the imaging device (e.g., the imaging device 110). More details may be found in operation 910 and the related descriptions thereof.

In some embodiments, the scanning parameter(s) corresponding to the current scan of the target subject may be different from the historical scanning parameter(s). That is, the current scanning parameter(s) of the target subject may be obtained by adjusting the historical scanning parameter(s) based on the information variation.

In 1020, a target image of the target subject may be obtained by processing the scan data based on one or more historical processing parameter(s). In some embodiments, operation 1020 may be performed by the processing device 120 or the data processing module 440.

In some embodiments, the processing device may obtain the target image of the target subject by processing an output of a detector based on one or more of historical processing parameters. Processing the scan data based on the historical processing parameter(s) may be similar to processing the scan data based on the current processing parameter(s) or preset processing parameter(s). More details may be found in operation 910 and the related descriptions thereof, which is not limited herein.

In some embodiments, the processing parameter(s) corresponding to the current scan of the target subject may be different from the historical processing parameter(s).

In 1030, a historical image may be obtained. In 1040, a comparison result may be obtained by comparing the target image of the target subject with the historical image. Operations 1030 and 1040 may be similar to operations 930 and 940 in the process 900. More details may be found in FIG. 9 and the related descriptions thereof, which is not limited herein.

It should be noted that the above description regarding process 1000 is merely provided for the purposes of illustration, and not intended to limit the scope of the present disclosure. For those skilled in the art, various modifications and variations may be made for the process 1000 under the guidance of the present disclosure. However, these modifications and variations do not depart from the scope of the present disclosure.

FIG. 11 is a flowchart illustrating an exemplary imaging process according to some embodiments of the present disclosure. In some embodiments, an imaging process 1100 may be executed by the imaging system 100 (e.g., the processing device 120), the computing device 200 (e.g., the processor 210), or the imaging system 400. For example, the imaging method 1100 may be implemented as a set of instructions or an application stored in a storage device (e.g., the storage device 140). The processing device 120 may execute the set of instructions or the application and may accordingly be directed to perform the imaging method 1100.

In 1110, scan data may be obtained by scanning a target subject based on one or more historical scanning parameters. In some embodiments, operation 1110 may be performed by the processing device 120 or the second scanning module 430.

The target subject may be scanned based on the historical scanning parameter(s). That is, the scanning parameter(s) corresponding to the current scan of the target subject may be the same as the historical scanning parameter(s). In some embodiments, the processing device may scan the target subject by performing a parameter setting based on the historical scanning parameter(s) and obtain the scan data. More details may be found in operation 910 and the related descriptions thereof.

In 1120, a target image of the target subject may be obtained by processing the scan data based on one or more current processing parameters. In some embodiments, operation 1120 may be performed by the processing device 120 or the data processing module 440.

In some embodiments, the processing device may obtain the target image of the target subject by processing an output of a detector based on one or more of current processing parameters. More details may be found in operation 920 and the related descriptions thereof.

In some embodiments, the processing parameter corresponding to the current scan of the target subject may be different from the historical processing parameter(s). That is, the current processing parameter(s) may be obtained by adjusting the historical processing parameter(s) based on the information variation.

In 1130, a historical image may be obtained. In 1140, a comparison result may be obtained by comparing the target image of the target subject with the historical image. Operations 1130 and 1140 may be similar to operations 930 and 940 in the process 900. More details may be found in FIG. 9 and the related descriptions thereof, which is not limited herein.

It should be noted that the above description regarding process 1100 is merely provided for the purposes of illustration, and not intended to limit the scope of the present disclosure. For those skilled in the art, various modifications and variations may be made for the process 1100 under the guidance of the present disclosure. However, these modifications and variations do not depart from the scope of the present disclosure.

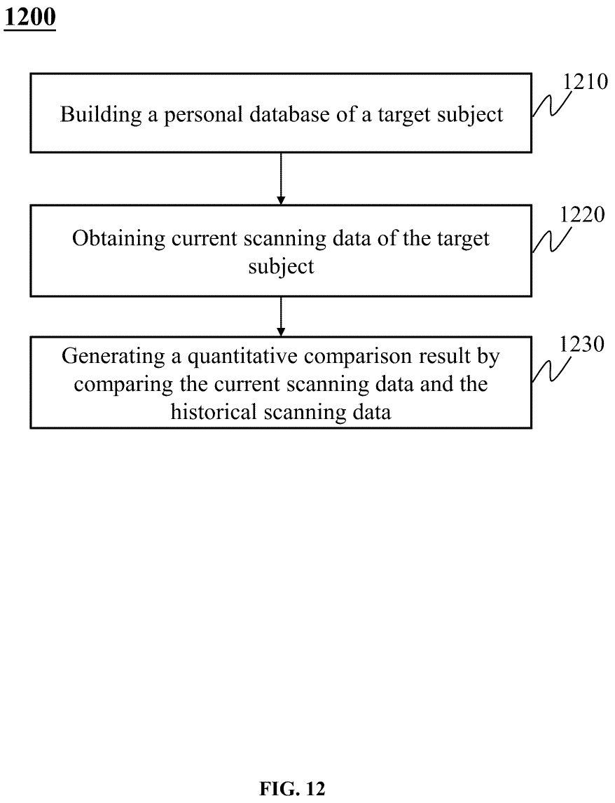

FIG. 12 is a flowchart illustrating an exemplary imaging process according to some embodiments of the present disclosure.

In some embodiments, an imaging process 1200 may be executed by the imaging system 100 (e.g., the processing device 120) or the computing device 200 (e.g., the processor 210). For example, the imaging method 1200 may be implemented as a set of instructions or an application stored in a storage device (e.g., the storage device 140). The processing device 120 may execute the set of instructions or the application and may accordingly be directed to perform the imaging method 1200.

In 1210, a personal database of a target subject may be built. In some embodiments, operation 1210 may be performed by the processing device 120.

In some embodiments, the personal database may be configured to store historical scanning data of the target subject. In some embodiments, historical scanning data may include at least one historical parameter (e.g., the historical scanning parameter and the historical processing parameter) of the target subject, at least one historical image of the target subject, and reference information of the target subject. The reference information may include information relating to at least one of a reference body shape, a reference body fat rate, or a reference 3D model of the target subject. More details about the historical parameter and the reference information may be found in FIGS. 5-11 and the related descriptions thereof, which are not repeated herein.

In some embodiments, the processing device 120 may build a personal database for each patient. For example, the processing device 120 may archive and store data such as the body shape, the body fat rate, the 3D model of the patient, or the like, the scanning parameter(s) used during the scan of the patient, the post-processing parameter(s) used after the scan, the scanning image, or the like, that correspond to the current scan in the personal database corresponding to the patient after the scan and/or the diagnosis of the patient is finished.

In 1220, current scanning data of the target subject may be obtained. In some embodiments, operation 1220 may be performed by the processing device 120.

In some embodiments, the current scanning data may include at least one current parameter (e.g., the current scanning parameter and the current processing parameter) of the target subject, at least one target image of the target subject, and current information of the target subject.

In some embodiments, a target image of the target subject may be obtained based on the at least one historical parameter and the reference information of the target subject, so that the target image may be comparable with the at least one historical image of the target subject.

In some embodiments, the processing device 120 may obtain current information of the target subject, determine at least one current parameter of the target subject based on the current information, the reference information, and the at least one historical parameter, and obtain the target image of the target subject based on the at least one current parameter. The current information may include information relating to at least one of a current body shape, a current body fat rate, or a current 3D model. More details may be found in FIGS. 5-8 and the related descriptions thereof, which are not repeated herein.

In 1230, a quantitative comparison result may be generated by comparing the current scanning data and the historical scanning data.

The comparison result may indicate a quantitative analysis result of material decomposition generated based on the comparison between the comparing the current image data and the historical image(s). More details may be found in FIGS. 9-11 and the related descriptions thereof, which are not repeated herein.

It should be noted that the above description regarding process 1200 is merely provided for the purposes of illustration, and not intended to limit the scope of the present disclosure. For those skilled in the art, various modifications and variations may be made for the process 1200 under the guidance of the present disclosure. However, these modifications and variations do not depart from the scope of the present disclosure.

One or more embodiments of the present disclosure also provide a non-transitory computer readable medium storing instructions. The instructions, when executed by at least one processor, may cause the at least one processor to implement a method (e.g., the process 500 to the process 1100) illustrated above.

One or more embodiments of the present disclosure also provide a system for imaging using a photon counting computed tomography device. The system may comprise at least one storage device storing a set of instructions, and at least one processor in communication with the storage device. When executing the set of instructions, the at least one processor may be configured to cause the system to perform operations 600.

One or more embodiments of the present disclosure also provide a system for imaging. The system may comprise at least one storage device storing a set of instructions, and at least one processor in communication with the storage device. When executing the set of instructions, the at least one processor may be configured to cause the system to perform operations 500.

Beneficial effects provided by the one or more embodiments of the present disclosure may include but not be limited to: (1) current scanning parameter(s) and/or current processing parameter(s) of a target subject may be determined based on current information, reference information, historical scanning parameter(s), historical processing parameter(s), etc., so that a certain correlation between scanning parameter(s) and processing parameter(s) during different scans of a same patient may be achieved; (2) the current scanning parameter(s) may be determined based on the historical scanning parameter(s) of the target subject, so that a quantitative comparability of clinic results in a series of scans of the patient may be achieved, thereby providing more reliable data support for a doctor to determine a condition of the patient; (3) a personal database storing related diagnosis data of different times of diagnoses may be established for each patient, so that a comparison of scanning states of the patient in different scans and a determination of optimal scanning parameter(s) and processing parameter(s) may be achieved to ensure a quantitative comparability of a scanning result of the patient before and after the scan; (4) a comparison result may be output by comparing the target image of the target subject with the historical image, so that a state change of the patient before and after the scan may be provided to the doctor quickly, thereby facilitating the diagnosis and disease treatment for the target subject.

It should be noted that the beneficial effect provided by different embodiments may be different. In different embodiments, a possible beneficial effect may be any one of the beneficial effect illustrated above, or a combination thereof, or a possible beneficial effect may be any beneficial effect that may be achieved.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures or characteristics may be combined as suitable in one or more embodiments of the present disclosure.

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, for example, an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various inventive embodiments. This method of disclosure, however, is not to be interpreted as reflecting an intention that the claimed object matter requires more features than are expressly recited in each claim. Rather, inventive embodiments lie in less than all features of a single foregoing disclosed embodiment.

In some embodiments, the numbers expressing quantities or properties used to describe and claim certain embodiments of the application are to be understood as being modified in some instances by the term “about,” “approximate,” or “substantially.” For example, “about,” “approximate,” or “substantially” may indicate ±1%, ±5%, ±10%, or ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that may be employed may be within the scope of the application. Thus, by way of example, but not of limitation, alternative configurations of the embodiments of the application may be utilized in accordance with the teachings herein. Accordingly, embodiments of the present application are not limited to that precisely as shown and described.

1. A method implemented on at least one machine each of which has at least one processor and at least one storage device for imaging, comprising:
obtaining current information of a target subject, the current information including information relating to current radiation attenuation of the target object;
obtaining reference information of the target subject, the reference information including information relating to historical radiation attenuation of the target object;
determining, based on the current information and the reference information, at least one current parameter of the target subject; and
obtaining, based on the at least one current parameter, a target image of the target subject.

2. The method of claim 1, wherein
the current information including information relating to at least one of a current body shape of the target subject, a current body fat rate of the target subject, or a current 3D model of the target object; and
the reference information including information relating to at least one of a reference body shape of the target subject, a reference body fat rate of the target subject, or a reference 3D model of the target object.

3. The method of claim 1, further comprising:
obtaining a comparison result by comparing the target image of the target subject with a historical image corresponding to the reference information.

4. The method of claim 1, the determining, based on the current information and the reference information, at least one current parameter of the target subject including:
obtaining at least one historical parameter corresponding to the reference information; and
determining, based on the current information, the reference information, and the at least one historical parameter, the at least one current parameter.

5. The method of claim 4, the determining, based on the current information, the reference information, and the at least one historical parameter, the at least one current parameter including:
obtaining information variation between the current information and the reference information, the information variation being related to radiation attenuation variation of the target subject; and
determining, based on the information variation and the at least one historical parameter, the at least one current parameter.

6. The method of claim 5, the determining, based on the information variation and the at least one historical parameter, the at least one current parameter including:
obtaining at least one initial parameter based on the at least one historical parameter;
updating the at least one initial parameter iteratively based on the information variation until a predicted image generated based on the at least one updated parameter and the historical image satisfy a preset condition; and
determining the at least one updated parameter as the least one current parameter.

7. The method of claim 6, wherein the preset condition includes:
the predicted image has a same or a substantially similar confidence degree with the historical image; or
a background region of the predicted image has at least one substantially same feature value as a background region of the historical image.

8. The method of claim 4, wherein
the at least one historical parameter includes at least one of a historical scanning parameter or a historical processing parameter; and
the at least one current parameter includes at least one of a current scanning parameter or a current processing parameter.

9. The method of claim 8, the determining, based on the current information, the reference information, and the at least one historical parameter, the at least one current parameter including:
determining the current scanning parameter based on the current information, the reference information, and the historical scanning parameter.

10. The method of claim 9, the obtaining, based on the at least one current parameter, a target image of the target subject including:
obtaining scan data by scanning the target subject based on the current scanning parameter; and
obtaining the target image of the target subject by processing the scan data based on the historical processing parameter.

11. The method of claim 8, the determining, based on the current information, the reference information, and the at least one historical parameter, the at least one current parameter including:
determining, based on the current information, the reference information, and the historical processing parameter, the current processing parameter.

12. The method of claim 11, the obtaining, based on the at least one current parameter, a target image of the target subject including:
obtaining scan data by scanning the target subject based on the historical scanning parameter; and
obtaining the target image of the target subject by processing the scan data based on the current processing parameter.

13. The method of claim 11, the obtaining, based on the at least one current parameter, a target image of the target subject including:
obtaining scan data by scanning the target subject based on the current scanning parameter or a preset scanning parameter; and
obtaining the target image of the target subject by processing the scan data based on the current processing parameter or a preset processing parameter.

14. A method implemented on at least one machine each of which has at least one processor and at least one storage device for image processing, comprising:
building a personal database of a target subject, the personal database being configured to store historical scanning data of the target subject, the historical scanning data including at least one historical parameter of the target subject, at least one historical image of the target subject, and reference information of the target subject, the reference information including information relating to historical radiation attenuation of the target object;
obtaining current scanning data of the target subject; and
generating a quantitative comparison result by comparing the current scanning data and the historical scanning data.

15-16. (canceled)

17. A method implemented on at least one machine each of which has at least one processor and at least one storage device for imaging using a photon counting computed tomography device, comprising:
obtaining current information of a target subject, the current information including information relating to current radiation attenuation of the target object;
obtaining reference information and at least one historical parameter of the target subject, the at least one historical parameter including at least one of a historical scanning parameter or a historical processing parameter, the reference information including information relating to historical radiation attenuation of the target object; and
determining, based on the current information, the reference information, and the at least one historical parameter, a current scanning parameter and/or a current processing parameter of the target subject.

18. The method of claim 17, further comprising:
obtaining an output of a detector of the photon counting computed tomography device by scanning the target subject based on the current scanning parameter or a preset scanning parameter; and
obtaining a target image of the target subject by processing the output of the detector based on the current processing parameter.

19. The method of claim 18, wherein the reference information and the at least one historical parameter are obtained from a personal database of the target subject, and the method further includes:
obtaining a historical image of the target subject from the personal database; and
comparing the target image with the historical image of the target subject and outputting a comparison result; and
wherein the target image includes a material decomposition image of the target subject, the target image includes a same or a substantially similar confidence degree with the historical image, and the confidence degree indicates an accuracy of the material decomposition image.

20. (canceled)

21. The method of claim 19, wherein the comparison result includes at least one of:
a comparison result between a current base material image and a historical base material image;
a comparison result between a current combined image and a historical combined image, the current combined image being obtained by combining a plurality of current base materials based on a first set of weights, and the historical combined image being obtained by combining a plurality of historical base materials based on a second set of weights, wherein the first set of weights is configured to make a numerical result of a non-target region of the current combined image be consistent with a numerical result of a non-target region of the historical combined image.

22. The method of claim 17, the determining, based on the current information, the reference information, and the at least one historical parameter, a current scanning parameter and/or a current processing parameter of the target subject including:
obtaining information variation by comparing the current information with the reference information of the target subject, the information variation being related to attenuation information of the target subject; and
determining, based on the information variation and the at least one historical parameter, the current scanning parameter and/or the current processing parameter of the target subject.

23. The method of claim 22, the determining, based on the information variation and the at least one historical parameter, the current scanning parameter and/or the current processing parameter of the target subject including:
converting the information variation into scanning parameter variation, and determining the current scanning parameter of the target subject based on the scanning parameter variation and the historical scanning parameter; and/or
converting the information variation into processing parameter variation, and determining the current processing parameter of the target subject based on the processing parameter variation and the historical processing parameter.

24-30. (canceled)
